(19) United States
(12) Patent Application Publication
(10) Pub. No.: US 2024/0100096 A1
(43) Pub. Date: Mar. 28, 2024

(54) METHODS OF ACTIVATING T CELLS

(71) Applicant: ADOC SSF, LLC, South San Francisco, CA (US)

(72) Inventors: Andrew Y. Hu, South San Francisco, CA (US); Ramya H. Tunuguntia, South San Francisco, CA (US)

(73) Assignee: ADOC SSF, LLC, South San Francisco, CA (US)

(21) Appl. No.: 18/535,741

(22) Filed: Dec. 11, 2023

Related U.S. Application Data
(63) Continuation of application No. PCT/US22/32936, filed on Jun. 10, 2022.
(60) Provisional application No. 63/209,784, filed on Jun. 11, 2021.

(51) Int. Cl. A61K 35/17; C12N 5/0783

(52) U.S. Cl. CPC A61K 35/17; C12N 5/0636; A61K 2039/5158; C12N 2501/2307; C12N 2501/2315; C12N 2501/51; C12N 2501/515

(57) ABSTRACT

The present disclosure provides novel artificial antigen presenting cells (aAPCs). The aAPCs disclosed herein comprise a liposome comprising a phospholipid and a stimulatory ligand displayed on the outer surface of the liposome. The aAPCs of the present disclosure can be used as an “off the shelf” tool to activate and expand a T cell of interest. Also, the present disclosure provides methods of activating a T cell and manufacturing a T cell therapy product using the aAPCs disclosed herein.

### CROSS-REFERENCE TO RELATED APPLICATIONS

This application is a Continuation application of International Patent Application No. PCT/US22/32936, filed Jun. 10, 2022, which claims benefit of priority to U.S. provisional application No. 63/209,784, filed 11 Jun. 2021, the contents of each which is incorporated herein by reference in its entirety and to each of which priority is claimed.

### BACKGROUND OF THE INVENTION

In the last decade cell therapies have emerged as a novel therapeutic for treating diseases. Specifically, the use of manufactured T cells (e.g., TILs, CAR-T and NeoTCR engineered cells) has become an area of increased interest. While many therapies are able to generate small scale, research grade results showing the effectiveness of such cell therapies for the treatment of cancer, a major limitation of getting these therapies to patients is manufacturing capabilities.

One critical step in the manufacture of cell therapies is in vitro activation of the cells. When cultivated in vitro, naive T cells gradually acquire the surface marker phenotypes of memory T cells following T cell receptor (TCR) stimulation, transitioning from stem cell-like memory (Tmsc) to central memory (Tcm) and finally to effector memory (Tem) T cells. Young T cells, particularly Tmsc cells, have demonstrated superior antitumor effects in multiple cancer immunotherapy models and show greater long-term survival when infused in vivo. Thus, in vitro expansion of antitumor T cells needs to be optimized to obtain efficient expansion while maintaining a Tmsc phenotype.

Repeated or overstimulation using highly immunogenic professional APCs, such as dendritic cells, unavoidably matures T cells and leads to T cell activation-induced cell death (AICD), especially for T cells that possess high antigen-specific avidity. Because of their highly potent immunogenicity, professional APCs are not the best choice for generating in vitro T-cells for use in cell therapy products. As a result, a variety of artificial antigen presenting cells or APC analogs (aAPCs) have been developed. For example, certain cell lines, such as K562, have been used. K562 is a human erythroleukemic cell line that was derived from a patient with chronic myelogenous leukemia in blastic crisis. K562 cells do not express endogenous HLA class I, II, or CD1d molecules but do express ICAM-1 (CD54) and LFA-3 (CD58), which are adhesion molecules required to form an effective immunological synapse. However, culturing and maintaining a population of K562 cells is costly and time consuming. Accordingly, a variety of non-cellular aAPCs have been developed and are commercially available. For example, microbeads or nanoparticles functionalized with activating antibodies for CD3 (αCD3) and CD28 (αCD28) are commonly used. However, because these commercial products covalently bind the activating antibodies to a solid-phase support, the activating molecules are static, unlike a natural antigen presenting cells where stimulatory ligands move within the cell membrane to enable TCR clustering, a key step in T cell activation.

Limitations of current technologies include: 1) interaction between the T cells and the activation particle are static and non-native, 2) beads or other inert particles can lead to chronic activation which results in overstimulated cells, 3) disparity in expansion of CD8+ and CD4+ T cells, 4) viability of CD8 T cells is poorer compared to CD4 cells after long periods of in vitro culture, and 5) there is lack of control of cytokine release.

The methods and compositions described herein aim to solve the problem and meet the unmet need of activating T cells in vitro for the manufacture of cell therapies for the treatment of patients.

### SUMMARY OF THE INVENTION

The present disclosure provides novel artificial antigen presenting cells (aAPCs) that can be used as an “off the shelf” tool to activate and expand a T cell of interest.

In certain embodiments, the present disclosure provides an artificial antigen presenting cell (aAPC) comprising a liposome comprising a phospholipid and a stimulatory ligand displayed on the outer surface of the liposome.

In certain embodiments, the stimulatory ligand is selected from the group consisting of a CD3 agonist, a CD28 agonist, a Major Histocompatibility Complex (MHC), a peptide-MHC complex, a multimerized neoepitope-HLA complex, CD58, CD86, CD83, 4-1BBL, OX40L, ICOSL (B7H2, B7RP1), CD40L, and an LFA-1. In certain embodiments, the liposome comprises a mixture of phospholipid and functionalized lipid.

In certain embodiments, a ratio of phospholipid to functionalized lipid in the mixture is between 10,000:1 and 25:1. In certain embodiments, the ratio is between 1000:1 and 50:1. In certain embodiments, the ratio is between 100:1 and 50:1.

In certain embodiments, the phospholipid is selected from the group consisting of phosphatidic acid (phosphatidate) (PA), phosphatidylethanolamine (cephalin) (PE), phosphatidylcholine (lecithin) (PC), phosphatidylserine (PS), a phosphoinositide, phosphatidylinositol (PI), phosphatidylinositol phosphate (PIP), phosphatidylinositol bisphosphate (PIP2), phosphatidylinositol triphosphate (PIP3), ceramide phosphorylcholine (Sphingomyelin) (SPH), ceramide phosphorylethanolamine (Sphingomyelin) (Cer-PE), and a combination thereof. In certain embodiments, the liposome comprises 18:1 palmitoyl-2-oleoyl-sn-glycero-3-phosphocholine (POPC) and/or 1-palmitoyl-2-oleoyl-sn-glycero-3-phosphoethanolamine (POPE). In certain embodiments, the functionalized lipid comprises a biotin moiety, a N-hydroxysuccinimide (NHS) moiety, a sulfo-NHS moiety, a nitrilotriacetic acid (NTA)-nickel, a maleimide moiety, or a N-benzylguanine. In certain embodiments, the functionalized lipid is a 1-oleoyl-2-(12-biotinyl-(aminododecanoyl))-sn-glycero-3-phosphoethanolamine (18:1-12:0 Biotin-PE), a 1,2-dipalmitoyl-sn-glycero-3-phosphoethanolamine-N-(biotinyl) (16:0 Biotin-PE), a 1,2-dioleoyl-sn-glycero-3-phosphoethanolamine-N-(biotinyl) (18:1 Biotin-PE), a 1,2-dioleoyl-sn-glycero-3-phosphoethanolamine-N-(cap biotinyl), (18:1 Biotin-Cap-PE), a 1,2-dipalmitoyl-sn-glycero-3-phosphoethanolamine-N-(cap biotinyl) (16:0 Biotin-Cap-PE), a biotin-Phosphatidylethanolamine (biotin-PE), or a biotin-1-palmitoyl-2-oleoyl-sn-glycero-3-phosphoethanolamine (biotin-POPE). In certain embodiments, the functionalized lipid is an 18:1 biotin-Cap-PE, a 16:0 biotin-Cap-PE, or a biotin-POPE. In certain embodiments, the functionalized lipid is a biotin-POPE.

In certain embodiments, the stimulatory ligand is attached to the liposome via the functionalized lipid. In certain embodiments, the stimulatory ligand is a CD3 agonist, a CD28 agonist, or a combination thereof. In certain embodiments, the CD3 agonist is an anti-CD3 antibody. In certain embodiments, the CD28 agonist is an anti-CD28 antibody. In certain embodiments, the anti-CD3 antibody and/or the anti-CD28 antibody is a low-endotoxin azide-free (LEAF) antibody.

In certain embodiments, the liposome has a diameter between 30 nm and 2 In certain embodiments, the liposome has a diameter between 50 nm and 600 nm. In certain embodiments, the liposome has a diameter between 100 nm and 400 nm.

In certain embodiments, the present disclosure provides a population of aAPCs disclosed herein. In certain embodiments, the liposomes of the population have a mean diameter between 30 nm and 2 μm and a size distribution of 5% to 50%. In certain embodiments, the mean diameter is between 50 nm and 600 nm. In certain embodiments, the mean diameter is between 100 nm and 400 nm.

In certain embodiments, the present disclosure provides a composition comprising a population of T cells and a population of artificial antigen presenting cells (aAPCs), wherein each aAPC comprises a liposome comprising a phospholipid and a stimulatory ligand displayed on the outer surface of the liposome. In certain embodiments, the liposome comprises a mixture of phospholipid and functionalized lipid.

In certain embodiments, a ratio of phospholipid to functionalized lipid in the mixture is between 10,000:1 and 25:1. In certain embodiments, the ratio is between 1000:1 and 50:1. In certain embodiments, the ratio is between 100:1 and 50:1.

In certain embodiments, the phospholipid is selected from the group consisting of phosphatidic acid (phosphatidate) (PA), phosphatidylethanolamine (cephalin) (PE), phosphatidylcholine (lecithin) (PC), phosphatidylserine (PS), a phosphoinositide, phosphatidylinositol (PI), phosphatidylinositol phosphate (PIP), phosphatidylinositol bisphosphate (PIP2), phosphatidylinositol triphosphate (PIP3), ceramide phosphorylcholine (Sphingomyelin) (SPH), ceramide phosphorylethanolamine (Sphingomyelin) (Cer-PE), and a combination thereof. In certain embodiments, the liposome comprises 18:1 palmitoyl-2-oleoyl-sn-glycero-3-phosphocholine (POPC) and/or 1-palmitoyl-2-oleoyl-sn-glycero-3-phosphoethanolamine (POPE). In certain embodiments, the functionalized lipid comprises a biotin moiety, a N-hydroxysuccinimide (NHS) moiety, a sulfo-NHS moiety, a nitrilotriacetic acid (NTA)-nickel, a maleimide moiety, or a N-benzylguanine. In certain embodiments, the functionalized lipid is a 1-oleoyl-2-(12-biotinyl-(aminododecanoyl))-sn-glycero-3-phosphoethanolamine (18:1-12:0 Biotin-PE), a 1,2-dipalmitoyl-sn-glycero-3-phosphoethanolamine-N-(biotinyl) (16:0 Biotin-PE), a 1,2-dioleoyl-sn-glycero-3-phosphoethanolamine-N-(biotinyl) (18:1 Biotin-PE), a 1,2-dioleoyl-sn-glycero-3-phosphoethanolamine-N-(cap biotinyl), (18:1 Biotin-Cap-PE), a 1,2-dipalmitoyl-sn-glycero-3-phosphoethanolamine-N-(cap biotinyl) (16:0 Biotin-Cap-PE), a biotin-Phosphatidylethanolamine (biotin-PE), or a biotin-1-palmitoyl-2-oleoyl-sn-glycero-3-phosphoethanolamine (biotin-POPE). In certain embodiments, the functionalized lipid is an 18:1 biotin-Cap-PE, a 16:0 biotin-Cap-PE, or a biotin-POPE. In certain embodiments, the functionalized lipid is a biotin-POPE.

In certain embodiments, the stimulatory ligand is attached to the liposome via the functionalized lipid. In certain embodiments, the stimulatory ligand is selected from the group consisting of a CD3 agonist, a CD28 agonist, a Major Histocompatibility Complex (MHC), a peptide-MHC complex, a multimerized neoepitope-HLA complex, CD58, CD86, CD83, 4-1BBL, OX40L, ICOSL (B7H2, B7RP1), CD40L, and an LFA-1. In certain embodiments, the stimulatory ligand is a CD3 agonist, a CD28 agonist, or a combination thereof. In certain embodiments, the CD3 agonist is an anti-CD3 antibody. In certain embodiments, the CD28 agonist is an anti-CD28 antibody. In certain embodiments, the anti-CD3 antibody and/or the anti-CD28 antibody is a low-endotoxin azide-free (LEAF) antibody.

In certain embodiments, the liposome has a diameter between 30 nm and 2 μm. In certain embodiments, the liposome has a diameter between 50 nm and 600 nm. In certain embodiments, the liposome has a diameter between 100 nm and 400 nm.

In certain embodiments, the composition further comprises a cell growth medium. In certain embodiments, further comprising interleukin 7 (IL-7) and interleukin 15 (IL-15). In certain embodiments, wherein the population of T cells comprises at least one NeoTCR cell.

In certain embodiments, the present disclosure provides a composition comprising a population of T cells and a population of artificial antigen presenting cells (aAPCs) disclosed herein.

In certain embodiments, the present disclosure provides a method of activating a T cell comprising exposing a T cell to one or more artificial antigen presenting cells (aAPCs), wherein each aAPC comprises a liposome comprising a phospholipid and a stimulatory ligand displayed on the outer surface of the liposome.

In certain embodiments, the phospholipid is selected from the group consisting of phosphatidic acid (phosphatidate) (PA), phosphatidylethanolamine (cephalin) (PE), phosphatidylcholine (lecithin) (PC), phosphatidylserine (PS), a phosphoinositide, phosphatidylinositol (PI), phosphatidylinositol phosphate (PIP), phosphatidylinositol bisphosphate (PIP2), phosphatidylinositol triphosphate (PIP3), ceramide phosphorylcholine (Sphingomyelin) (SPH), ceramide phosphorylethanolamine (Sphingomyelin) (Cer-PE), and a combination thereof. In certain embodiments, the liposome comprises 18:1 palmitoyl-2-oleoyl-sn-glycero-3-phosphocholine (POPC) and/or 1-palmitoyl-2-oleoyl-sn-glycero-3-phosphoethanolamine (POPE).

In certain embodiments, the stimulatory ligand is selected from the group consisting of a CD3 agonist, a CD28 agonist, a Major Histocompatibility Complex (MHC), a peptide-MHC complex, a multimerized neoepitope-HLA complex, CD58, CD86, CD83, 4-1BBL, OX40L, ICOSL (B7H2, B7RP1), and CD40L. In certain embodiments, the stimulatory ligand is a CD3 agonist, a CD28 agonist, or a combination thereof. In certain embodiments, the CD3 agonist is an anti-CD3 antibody. In certain embodiments, the CD28 agonist is an anti-CD28 antibody. In certain embodiments, the anti-CD3 antibody and/or the anti-CD28 antibody is a low-endotoxin azide-free (LEAF) antibody.

In certain embodiments, the method further comprises mixing a population of T cells with a population of aAPCs. In certain embodiments, wherein the liposomes of the population of aAPCs have a mean diameter between 30 nm and 2 μm and a size distribution of 5% to 50%. In certain embodiments, the mean diameter is between 30 nm and 400 nm. In certain embodiments, the mean diameter is approximately 200 nm. In certain embodiments, the mixture comprises aAPCs and T cells in a ratio of between 5:1 (aAPCs:T cells) and 5000:1. In certain embodiments, the T cell is a NeoTCR cell.

In certain embodiments, the present disclosure provides a method of manufacturing a T cell therapy product comprising exposing a population of T cells to a population of artificial antigen presenting cells (aAPCs), wherein each aAPC comprises a liposome comprising a phospholipid and a stimulatory ligand displayed on the outer surface of the liposome.

In certain embodiments, the method further comprises gene editing of at least one T cell of the population of T cells. In certain embodiments, the gene editing comprises electroporating the population of T cells with a dual ribonucleoprotein species of CRISPR-Cas9 nucleases bound to guide RNA sequences, wherein each species targets an endogenous TCRα locus and/or an endogenous TCRβ locus. In certain embodiments, the exposing occurs prior to the gene editing. In certain embodiments, the gene editing is non-viral. In certain embodiments, the population of T cells comprises one or more NeoTCR cells.

In certain embodiments, the present disclosure provides a method of treating a patient in need thereof with a T cell therapy, wherein the T cell therapy is obtained by the methods of manufacturing disclosed herein.

### BRIEF DESCRIPTION OF THE DRAWINGS

FIG. 1 shows a workflow of an aAPC with surface displayed anti-CD3 and anti-CD28.

FIGS. 2A and 2B show the monitoring of T cell clustering during activation phase.

FIGS. 3A-3D show the monitoring of T cell clustering during activation phase responsive to exposure to various aAPCs.

FIG. 4 shows that 0.1-2% POPE show increased % Tmsc and 4% POPE was comparable % Tmsc to TransAct.

FIG. 5 shows that increased dosage of stimulatory ligands increases Ttm/Tem populations and reduces Tmsc+Tcm populations.

### DETAILED DESCRIPTION

The present disclosure provides compositions and methods including artificial antigen presenting cells (aAPCs) useful for the preparation and manufacturing of adoptive cell therapies. The present disclosure is based, in part, on the ability of the inventors to create aAPCs comprising a liposome including a phospholipid and a stimulatory ligand. These aAPCs can be used as an “off the shelf” tool to activate and expand a T cell of interest. Finally, the present disclosure also provides methods for producing adoptive cell therapies (e.g., T cell therapy products) using the compositions and methods disclosed herein. Non-limiting embodiments of the present disclosure are described by the present description and examples. For purposes of clarity of disclosure and not by way of limitation, the detailed description is divided into the following subsections:
1. Definitions;
2. Artificial Antigen Presenting Cells;
3. T Cell Activation;
4. NeoTCR Products; and
5. Exemplary Embodiments.

### 1. Definitions

Unless defined otherwise, all technical and scientific terms used herein have the meaning commonly understood by a person skilled in the art. The following references provide one of skill with a general definition of many of the terms used in the presently disclosed subject matter: Concise Medical Dictionary, edited by Law and Martin, Oxford University Press, 2020; A Dictionary of Biology, edited by Hine, Oxford University Press, 2019; A Dictionary of Chemistry, edited by Law and Rennie, Oxford University Press, 2020; Oxford Dictionary of Biochemistry and Molecular Biology, edited by Cammack, Atwood, Campbell, Parish, Smith, Vella, and Stirling, Oxford University Press, 2006; and Paul, William. 2013. Fundamental Immunology. Philadelphia, PA: Wolters Kluwer Health/Lippincott Williams & Wilkins. As used herein, the following terms have the meanings ascribed to them below, unless specified otherwise.

It is understood that aspects and embodiments of the invention described herein include “comprising,” “consisting,” and “consisting essentially of” aspects and embodiments. The terms “comprises” and “comprising” are intended to have the broad meaning ascribed to them in U.S. Patent Law and can mean “includes”, “including” and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

The term “antibody” as used herein is used in the broadest sense and encompasses various antibody structures, including but not limited to monoclonal antibodies, polyclonal antibodies, multi-specific antibodies (e.g., bispecific and tri-specific antibodies), and antibody fragments (e.g., bis-Fabs) so long as they exhibit the desired antigen-binding activity. “Antibody Fragment” as used herein refers to a molecule other than an intact antibody that comprises a portion of an intact antibody that binds the antigen to which the intact antibody binds. Examples of antibody fragments include but are not limited to bis-Fabs; Fv; Fab; Fab, Fab′-SH; F(ab′)2; diabodies; linear antibodies; single-chain antibody molecules (e.g., scFv); and multi-specific antibodies formed from antibody fragments.

The terms “Cancer” and “Tumor” are used interchangeably herein. As used herein, the terms “Cancer” or “Tumor” refer to all neoplastic cell growth and proliferation, whether malignant or benign, and all pre-cancerous and cancerous cells and tissues. The terms are further used to refer to or describe the physiological condition in mammals that is typically characterized by unregulated cell growth/proliferation. Cancer can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Cancer includes cancers, such as sarcomas, carcinomas, or plasmacytomas (malignant tumor of the plasma cells). Examples of cancer include, but are not limited to, those described herein. The terms “Cancer” or “Tumor” and “Proliferative Disorder” are not mutually exclusive as used herein.

“Treat,” “treatment,” and “treating” are used interchangeably and as used herein mean obtaining beneficial or desired results including clinical results. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the NeoTCR Product of the invention are used to delay development of a proliferative disorder (e.g., cancer) or to slow the progression of such disease.

“Dextramer” as used herein means a multimerized neoepitope-HLA complex that specifically binds to its cognate NeoTCR.

As used herein, the terms “neoantigen”, “neoepitope” or “neoE” refer to a newly formed antigenic determinant that arises, e.g., from a somatic mutation(s) and is recognized as “non-self” A mutation giving rise to a “neoantigen”, “neoepitope” or “neoE” can include a frameshift or non-frameshift indel, missense or nonsense substitution, splice site alteration (e.g., alternatively spliced transcripts), genomic rearrangement or gene fusion, any genomic or expression alterations, or any post-translational modifications.

“NeoTCR” and “NeoE TCR” as used herein mean a neoepitope-specific T cell receptor that is introduced into a T cell, e.g., by gene editing methods.

“NeoTCR cells” as used herein means one or more cells precision engineered to express one or more NeoTCRs. In certain embodiments, the cells are T cells. In certain embodiments, the T cells are CD8+ and/or CD4+ T cells. In certain embodiments, the CD8+ and/or CD4+ T cells are autologous cells from the patient for whom a NeoTCR Product will be administered. The terms “NeoTCR cells,” “NeoTCR-P1 T cells” and “NeoTCR-P1 cells” are used interchangeably herein.

“NeoTCR Product” as used herein means a pharmaceutical formulation comprising one or more NeoTCR cells. NeoTCR Product consists of autologous precision genome-engineered CD8+ and/or CD4+ T cells. Using a targeted DNA-mediated non-viral precision genome engineering approach, expression of the endogenous TCR is eliminated and replaced by a patient-specific NeoTCR isolated from peripheral CD8+ T cells targeting the tumor-exclusive neoepitope. In certain embodiments, the resulting engineered CD8+ or CD4+ T cells express NeoTCRs on their surface of native sequence, native expression levels, and native TCR function. The sequences of the NeoTCR external binding domain and cytoplasmic signaling domains are unmodified from the TCR isolated from native CD8+ T cells. Regulation of the NeoTCR gene expression is driven by the native endogenous TCR promoter positioned upstream of where the NeoTCR gene cassette is integrated into the genome. Through this approach, native levels of NeoTCR expression are observed in unstimulated and antigen-activated T cell states.

The NeoTCR Product manufactured for each patient represents a defined dose of autologous CD8+ and/or CD4+ T cells that are precision genome engineered to express a single neoE-specific TCR cloned from neoE-specific CD8+ T cells individually isolated from the peripheral blood of that same patient.

“NeoTCR Viral Product” as used herein has the same definition of NeoTCR Product except that the genome engineering is performed using viral mediated methods.

“Pharmaceutical Formulation” refers to a preparation which is in such form as to permit the biological activity of an active ingredient contained therein to be effective, and which contains no additional components which are unacceptably toxic to a subject to which the formulation would be administered. For clarity, DMSO at quantities used in a NeoTCR Product is not considered unacceptably toxic.

A “subject,” “patient,” or an “individual” for purposes of treatment refers to any animal classified as a mammal, including humans, domestic and farm animals, and zoo, sports, or pet animals, such as dogs, horses, cats, cows, etc. Preferably, the mammal is human.

“TCR” as used herein means T cell receptor.

The term “endogenous” as used herein refers to a nucleic acid molecule or polypeptide that is normally expressed in a cell or tissue.

The term “exogenous” as used herein refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid that is not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

“Young” or “Younger” or “Young T cell” as it relates to T cells means memory stem cells (Tmsc) and central memory cells (Tcm). These cells have T cell proliferation upon specific activation and are competent for multiple cell divisions. They also have the ability to engraft after re-infusion, rapidly differentiate into effector T cells upon exposure to their cognate antigen and target and kill tumor cells, as well as persist for ongoing cancer surveillance and control.

Later in the continuum of T-cell differentiation and maturation are two antigen-experienced subtypes: effector memory T cells (Tem) and terminally differentiated effector T cells (Teff).

### 2. Artificial Antigen Presenting Cells

The present disclosure provides artificial antigen presenting cells for the preparation and manufacturing of adoptive cell therapies. Artificial antigen presenting cells (aAPCs) of the present disclosure were designed as lipid vesicles or lipid nanoparticles that are capable of displaying different agents that can be used to activate CD4 and CD8 T cells. The key parameters and considerations used to design the aAPCs are provided in Table 1.

TABLE 1
Considerations used for the design of aAPCs

| Key | Activated | |
|---|---|---|
| parameter | Biological APC | Liposomal aAPC |
| Signal 1: | pMHC | comPACT, tetramer, anti-CD3 |
| recognition | | mAb, other stimulatory ligands |
| Signal 2: | B7.1 and B7.2 | Presentation of soluble and |
| co-stimulation | | insoluble cues, anti-CD28 mAb, |
| | | 41BBL, OX40L, ICAM |
| Signal 3: | IL-2, chemokine | Sustained and localized release |
| secretable | CCL3, CCL4 | |
| signals | | |
| Immunological | membrane- | membrane-membrane |
| Synapse | membrane | |
| Size | 10-20 μm in diameter | 30 nm-10 μM in diameter |
| Shape | Long thin sheet like | Spherical, tubular, conformable |
| | projections | |

One key goal of using aAPCs was to create an activation agent with mobile ligands Immobile ligands (e.g., bead-bound or plate-coated reagents) are not ideal for T cell activation platforms. In contrast, long range membrane diffusivity allows natural movement of ligands which is ideal to mimic natural T cell activation and a more natural interaction with T cells. In view of this, it was necessary to experiment with a variety of different lipid compositions (i.e., different lipid combinations and different rations of the combinations) in order to find the composition that had the correct degree of fluidity (diffusivity) to allow for optimal T cell activation for the manufacture of cell therapies.

One key consideration for the design of the aAPCs was the known fact that T cell clustering impacts signal transduction and activation. The goal was to design aAPCs that could offer membrane fluidity to enable protein rearrangement on the surface.

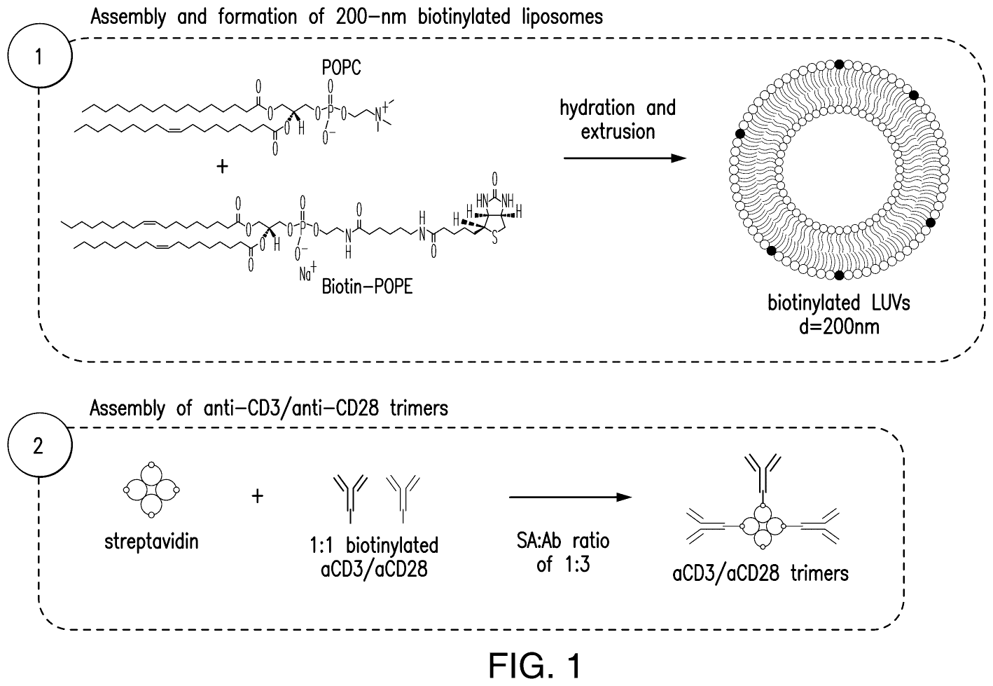

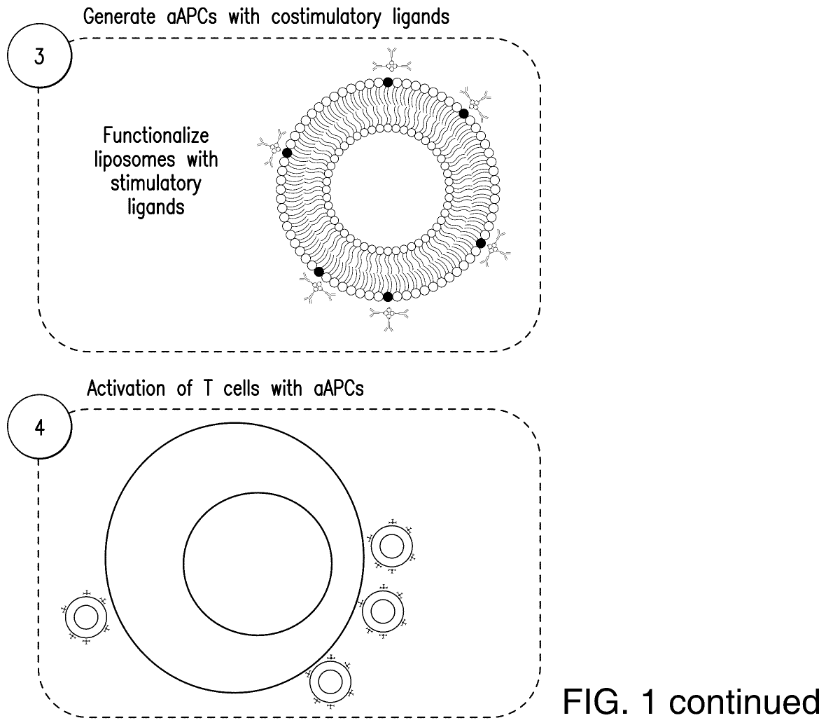

An example of the aAPC synthesis workflow of an αCD3/αCD28 aAPC is provided in FIG. 1. As shown, the two lipids used in this aAPC are POPC and POPE, wherein the POPE is biotinylated for the attachment of anti-CD3 and anti-CD28 antibodies, or other stimulatory ligands.

Proof of concept experiments were performed at small scale with aAPCs. These proof-of-concept experiments: 1) confirmed that the aAPCs of the invention are not toxic to T cells, 2) defined a range of acceptable density of ligand display, 3) defined a range of dosage (aAPC:cell), and 4) confirmed that stimulatory ligands can be displayed on the APCs via a biotin to streptavidin to biotin linkage. Liposomes do not adversely affect T cell proliferation and viability. The ratio of liposomes to T cells can vary from 25:1 to 10,000:1. More preferably the ratio is 100:1, 250:1, 500:1, 1000:1, 2000:1, 3000:1, 4000:1 or 5000:1. The ratio of aAPC to T cell and mean diameter of the aAPCs can vary inversely to maintain the same effect, i.e. larger liposomes can be provided at lower dosage than smaller liposomes to provide the same amount of T activation.

In certain implementations, it may be desirable to present more than one stimulatory ligand to induce activation of a T cell. The plurality of stimulatory ligands can be presented as coupled on a single type of aAPC (e.g. an aAPC displaying both αCD3 and αCD28) or uncoupled on different types of aAPC (e.g., one aAPC displaying only αCD3 and a second aAPC displaying only αCD28). Similarly, any particular stimulatory ligand can be displayed at equal concentrations or varying concentrations relative to simultaneously displayed stimulatory ligands.

Antigen Presenting Cells (APCs) provide signals to activate T cells in a natural, biological setting. APCs direct naïve T cells using three (3) main types of signals: 1) pMHC:TCR, 2) co-stimulation through cell surface proteins, and 3) T cell fate determination by cytokines.

2.1. Liposomes.

In addition to the methods and procedures exemplified herein, various methods routinely used by the skilled artisans for preparing liposomes can also be employed in the present invention. For example, the methods described in Chen et al., Blood 115:4778-86, 2010; and Liposome Technology, vol. 1, 2nd edition (by Gregory Gregoriadis (CRC Press, Boca Raton, Ann Arbor, London, Tokyo), Chapter 4, pp 67-80, Chapter 10, pp 167-184 and Chapter 17, pp 261-276 (1993)) can be used. More specifically, suitable methods include, but are not limited to, a sonication method, an ethanol injection method, a French press method, an ether injection method, a cholic acid method, a calcium fusion method, a lyophilization method and a reverse phase evaporation method. The structure of the liposome is not particularly limited, and may be any liposome such as unilamella and multilamella.

The disclosed liposomes disclosed herein typically include one or a combination of two or more lipids that can be neutral, anionic, or cationic at physiologic pH. The vesicles include, or otherwise can be formed from, any suitable lipid or combination of lipids. Likewise, the conjugates can include or otherwise be formed of any suitable lipid. In some embodiments, a combination of two, three, four, five, or more different lipid conjugates (e.g., different lipids and the same target moiety, different lipids and different targeting moieties, or the same lipid and different targeting moiety) can be inserted or otherwise added to the same vesicle.

The lipid or lipid-forming materials used to carry out the invention include all known materials for liposome or vesicle formation. Examples of useful materials include combinations of phospholipid molecules and cholesterol. Example phospholipid molecules include phosphatidic acid (phosphatidate) (PA), phosphatidylethanolamine (cephalin) (PE), phosphatidylcholine (lecithin) (PC), phosphatidylserine (PS), a phosphoinositide, phosphatidylinositol (PI), phosphatidylinositol phosphate (PIP), phosphatidylinositol bisphosphate (PIP2), phosphatidylinositol triphosphate (PIP3), ceramide phosphorylcholine (Sphingomyelin) (SPH), ceramide phosphorylethanolamine (Sphingomyelin) (Cer-PE). Particularly preferred are combinations comprising 18:1 palmitoyl-2-oleoyl-sn-glycero-3-phosphocholine (POPC).

Liposome compositions can be produced using the described methods, having mean diameters from 30 nm to 2000 nm (2 μm), e.g., 30 nm, 40 nm, 50 nm, 60 nm, 80 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 1.2 μm, 1.5 μm and 2 μm, and a size distribution of 5 to 50%, 10 to 30% or 15 to 20%. Preferably the liposome compositions have a mean diameter of between 50 nm and 600 nm, more preferably between 100 nm and 400 nm. The methods described here can be used to provide vesicles for activating T cells during manufacture of cell therapies.

2.2. Functionalized Lipids.

According to an embodiment of the present invention, a fraction of lipid forming the aAPC comprises a functional element conjugated to or otherwise linked, directly or indirectly, to the lipid. The functional element can be a reactive moiety, a small molecule, protein or polypeptide, carbohydrate, nucleic acid or a combination thereof. In preferred embodiments, at least one of the functional elements is a targeting moiety that increases attachment, binding, or association of the functionalized lipid vesicle to a target cell(s), tissues(s), and/or microenvironment(s) relative to the lipid vesicle. In certain implementations, a fraction of the lipid forming is a lipid functionalized with a reactive ligand. Specific examples of the suitable reactive moieties, the reacting ligands, and of the functionalized lipids containing are listed in Table 2.

TABLE 2
Exemplary Functionalized Lipids

| Reactive | Reacting | |
|---|---|---|
| Moiety | Moiety | Example functionalized Lipid |
| Biotin | Avidin, | 1-oleoyl-2-(12-biotinyl-(aminodo- |
| | streptavidin | decanoyl))-sn-glycero-3-phospho- |
| | | ethanolamine (18:1-12:0 Biotin PE); |
| | | 1,2-dipalmitoyl-sn-glycero-3-phospho- |
| | | ethanolamine-N-(biotinyl), 16:0 |
| | | Biotinyl PE |
| | | 1,2-dioleoyl-sn-glycero-3-phospho- |
| | | ethanolamine-N-(biotinyl), 18:1 |
| | | Biotinyl PE |
| | | 1,2-dioleoyl-sn-glycero-3-phospho- |
| | | ethanolamine-N-(cap |
| | | biotinyl), 18:1 Biotinyl Cap PE; |
| | | 1,2-dipalmitoyl-sn-glycero-3- |
| | | phosphoethanolamine-N-(cap |
| | | biotinyl), 16:0 Biotinyl Cap PE |
| N- | Amine | NHS Palmitic acid N-hydroxy- |
| hydroxy- | | succinimide ester |
| succinimide | | |
| (NHS), | | |
| Sulfo-NHS | | |
| Nitrilotriacetic | Histidine, | 1,2-dioleoyl-sn-glycero-3-[(N-(5-amino- |
| acid (NTA)- | His tags | 1-carboxypentyl) iminodiacetic acid) |
| nickel | | succinyl], 18:1 DGS-NTA (Ni) |
| Maleimide, e.g. | Thiol, e.g. | 1,2-dipalmitoyl-sn-glycero-3-phospho- |
| | thiolated | ethanolamine-N-[4-(p-maleimidomethyl) |
| | antibody | cyclohexane-carboxamide] (sodium salt), |
| | | 16:0 PE MCC; |
| | | 1,2-dioleoyl-sn-glycero-3-phospho- |
| | | ethanolamine-N-[4-(p-maleimidomethyl) |
| | | cyclohexane-carboxamide] (sodium salt), |
| | | 18:1 PE MCC; |
| | | 1,2-dioleoyl-sn-glycero-3-phosphocholine |
| | | (N-aminoethyl), 18:1 aminoethyl PC; |
| | | 1,2-dioleoyl-sn-glycero-3-phospho- |
| | | ethanolamine-N-[4-(p-maleimidophenyl) |
| | | butyramide] (sodium salt), 18:1 MPB PE |
| | | 1,2-dipalmitoyl-sn-glycero-3-phospho- |
| | | ethanolamine-N-[4-(p-maleimidophenyl) |
| | | butyramide] (sodium salt), 16:0 MPB PE |
| N-benzylguanine | SNAP-tag | 1,2-dioleoyl-sn-glycero-3-phospho- |
| | | ethanolamine-N-benzylguanine, |
| | | 18:1 PE-benzylguanine |
| | | 1,2-dioleoyl-sn-glycero-3-phospho- |
| | | ethanolamine-N-[benzylguanine |
| | | (polyethylene glycol)-2000], 18:1 PE- |
| | | PEG2000-benzylguanine |

Preferably, the reactive ligand is selected from biotin, N-hydroxysuccinimide (NHS) ester, sulfo-NHS ester, nitrilotriacetic acid (NTA)-nickel, amine, maleimides, dithiopyridinyl, pyridyl disulfide, pyridyldithiopropionate, and N-benzylguanine. Sulfhydryls, also called thiols, exist in proteins in the side-chain of cysteine (Cys, C) amino acids. Sulfhydryl-reactive chemical groups include haloacetyls, maleimides, aziridines, acryloyls, acylating agents, vinylsulfones, pyridyl disulfides, TNB-thiols and disulfide reducing agents.

Different lipids which are offered for thioether conjugation contain maleimide, aromatic maleimides such as N-[4-(p-maleimidophenyl)-butyryl] (MPB) or 4-(N-maleimidomethyl)cyclohexane-1-carboxylate (MCC) group. The maleimide function group of MCC which contains an aliphatic cyclohexane ring is more stable toward hydrolysis in aqueous reaction environments rather than the aromatic phenyl group of MPB. Conjugating a protein or polypeptide to a functionalized lipid can be performed in accordance with methods well known in the art. See, e.g., Chemistry of protein conjugation and cross-linking, Shan Wong, CRC Press (Boca Raton, Fla., 1991); and Bioconjugate techniques, 2nd ed., Greg T. Flermanson, Academic Press (London, U K, 2008). Alternatively, the stimulatory ligand, such as anti-CD3 and/or anti-CD28, can be attached via non-covalent means including biotin-streptavidin interactions.

### 3. T Cell Activation

In in vitro settings for the manufacture of T cells for cell therapy products, T cells need to be stimulated in order for them to expand. Cell expansion is critical for cell therapy development and manufacture because the T cells need to be able to proliferate in an in vitro culture in order to yield a cell product with a sufficient number of cells to be therapeutically beneficial for patients. More specifically, T cell activation determines the extent of in vitro cell proliferation (i.e., yield of cell product) and T cell differentiation (i.e., quality of the cell product). T cells are stimulated in vitro using antigen independent stimulation which can be mitogen driven. Such stimulation may include two main signals: 1) Signal 1, an anti-CD3 agent will bind to the CD3 chain of a TCR complex, and 2) Signal 2, an anti-CD28 gent will bind to CD28 on the T cells.

Commercially available products for polyclonal T cell expansion include superparamagnetic particles (e.g., TransAct, Dynabeads, ProMag Bind0IT, MagMax, and Spherotech), polymeric complexes that are either embedded or displayed on the surface (e.g., Cloudz), and soluble tetrameric antibody complexes (e.g., ImmunoCult). Two primary limitations of these commercially available products are: 1) the interaction between the T cells and activation product is non-native, and 2) the beads/inert particles of the activation products can lead to chronic activation which results in the overstimulation of the T cells.

Additional problems with the commercially available products include: 1) they are not tunable to drive the desired T cell characteristics for individual cell therapies, 2) they often vary in composition and activity by lots, 3) they are often not able to provide the activation needed to manufacture cell products at therapeutically relevant cell numbers, and 4) they are very expensive.

### 4. NeoTCR Products

In some embodiments, using the gene editing technology and NeoTCR isolation technology described in PCT/US2020/17887 and PCT/US2019/025415, which are incorporated herein in their entireties, NeoTCRs are cloned in autologous CD8+ and CD4+ T cells from the same patient with cancer by precision genome engineered to express the NeoTCR. In other words, the NeoTCRs that are tumor specific are identified in cancer patients, such NeoTCRs are then cloned, and then the cloned NeoTCRs are inserted into the cancer patient's own T cells. NeoTCR expressing T cells are then expanded in a manner that preserves a “young” T cell phenotypes, resulting in a NeoTCR-P1 product (i.e., a NeoTCR Product) in which the majority of the T cells exhibit T memory stem cell and T central memory phenotypes.

These ‘young’ or ‘younger’ or less-differentiated T cell phenotypes are described to confer improved engraftment potential and prolonged persistence post-infusion. Thus, the administration of NeoTCR Product, consisting significantly of ‘young’ T cell phenotypes, has the potential to benefit patients with cancer, through improved engraftment potential, prolonged persistence post-infusion, and rapid differentiation into effector T cells to eradicate tumor cells throughout the body.

Ex vivo mechanism-of-action studies were also performed with NeoTCR Products manufactured with T cells from patients with cancer. Comparable gene editing efficiencies and functional activities, as measured by antigen-specificity of T cell killing activity, proliferation, and cytokine production, were observed demonstrating that the manufacturing process described herein is successful in generating products with T cells from patients with cancer as starting material.

In certain embodiments, the NeoTCR Product manufacturing process involves electroporation of dual ribonucleoprotein species of CRISPR-Cas9 nucleases bound to guide RNA sequences, with each species targeting the genomic TCRα and the genomic TCRβ loci. The specificity of targeting Cas9 nucleases to each genomic locus has been previously described in the literature as being highly specific. Comprehensive testing of the NeoTCR Product was performed in vitro and in silico analyses to survey possible off-target genomic cleavage sites, using COSMID and GUIDE-seq, respectively. Multiple NeoTCR Products or comparable cell products from healthy donors were assessed for cleavage of the candidate off-target sites by deep sequencing, supporting the published evidence that the selected nucleases are highly specific.

Further aspects of the precision genome engineering process have been assessed for safety. No evidence of genomic instability following precision genome engineering was found in assessing multiple NeoTCR Products by targeted locus amplification (TLA) or standard FISH cytogenetics. No off-target integration anywhere into the genome of the NeoTCR sequence was detected. No evidence of residual Cas9 was found in the cell product.

The comprehensive assessment of the NeoTCR Product and precision genome engineering process indicates that the NeoTCR Product will be well tolerated following infusion back to the patient.

The genome engineering approach described herein enables the highly efficient generation of bespoke NeoTCR cells (i.e., NeoTCR Products) for personalized adoptive cell therapy for patients with solid and liquid tumors. Furthermore, the engineering method is not restricted to the use in T cells and has also been applied successfully to other primary cell types, including natural killer and hematopoietic stem cells.

### 5. Exemplary Embodiments

In certain embodiments, the present disclosure provides an artificial antigen presenting cell (aAPC) comprising a liposome comprising a phospholipid and a stimulatory ligand displayed on the outer surface of the liposome.

In certain embodiments of the aAPCs disclosed herein, the stimulatory ligand is selected from the group consisting of a CD3 agonist, a CD28 agonist, a Major Histocompatibility Complex (MHC), a peptide-MHC complex, a multimerized neoepitope-HLA complex, CD58, CD86, CD83, 4-1BBL, OX40L, ICOSL (B7H2, B7RP1), CD40L, and an LFA-1. In certain embodiments of the aAPCs disclosed herein, the liposome comprises a mixture of phospholipid and functionalized lipid. In certain embodiments of the aAPCs disclosed herein, a ratio of phospholipid to functionalized lipid in the mixture is between 10,000:1 and 25:1. In certain embodiments of the aAPCs disclosed herein, the ratio is between 1000:1 and 50:1. In certain embodiments of the aAPCs disclosed herein, the ratio is between 100:1 and 50:1.

In certain embodiments of the aAPCs disclosed herein, the phospholipid is selected from the group consisting of phosphatidic acid (phosphatidate) (PA), phosphatidylethanolamine (cephalin) (PE), phosphatidylcholine (lecithin) (PC), phosphatidylserine (PS), a phosphoinositide, phosphatidylinositol (PI), phosphatidylinositol phosphate (PIP), phosphatidylinositol bisphosphate (PIP2), phosphatidylinositol triphosphate (PIP3), ceramide phosphorylcholine (Sphingomyelin) (SPH), ceramide phosphorylethanolamine (Sphingomyelin) (Cer-PE), and a combination thereof. In certain embodiments of the aAPCs disclosed herein, the liposome comprises 18:1 palmitoyl-2-oleoyl-sn-glycero-3-phosphocholine (POPC) and/or 1-palmitoyl-2-oleoyl-sn-glycero-3-phosphoethanolamine (POPE). In certain embodiments of the aAPCs disclosed herein, the functionalized lipid comprises a biotin moiety, a N-hydroxysuccinimide (NHS) moiety, a sulfo-NHS moiety, a nitrilotriacetic acid (NTA)-nickel, a maleimide moiety, or a N-benzylguanine. In certain embodiments of the aAPCs disclosed herein, the functionalized lipid is a 1-oleoyl-2-(12-biotinyl-(aminododecanoyl))-sn-glycero-3-phosphoethanolamine (18:1-12:0 Biotin-PE), a 1,2-dipalmitoyl-sn-glycero-3-phosphoethanolamine-N-(biotinyl) (16:0 Biotin-PE), a 1,2-dioleoyl-sn-glycero-3-phosphoethanolamine-N-(biotinyl) (18:1 Biotin-PE), a 1,2-dioleoyl-sn-glycero-3-phosphoethanolamine-N-(cap biotinyl), (18:1 Biotin-Cap-PE), a 1,2-dipalmitoyl-sn-glycero-3-phosphoethanolamine-N-(cap biotinyl) (16:0 Biotin-Cap-PE), a biotin-Phosphatidylethanolamine (biotin-PE), or a biotin-1-palmitoyl-2-oleoyl-sn-glycero-3-phosphoethanolamine (biotin-POPE).

In certain embodiments of the aAPCs disclosed herein, the functionalized lipid is an 18:1 biotin-Cap-PE, a 16:0 biotin-Cap-PE, or a biotin-POPE. In certain embodiments of the aAPCs disclosed herein, the functionalized lipid is a biotin-POPE.

In certain embodiments of the aAPCs disclosed herein, the stimulatory ligand is attached to the liposome via the functionalized lipid. In certain embodiments of the aAPCs disclosed herein, the stimulatory ligand is a CD3 agonist, a CD28 agonist, or a combination thereof. In certain embodiments of the aAPCs disclosed herein, the CD3 agonist is an anti-CD3 antibody. In certain embodiments of the aAPCs disclosed herein, the CD28 agonist is an anti-CD28 antibody. In certain embodiments of the aAPCs disclosed herein, the anti-CD3 antibody and/or the anti-CD28 antibody is a low-endotoxin azide-free (LEAF) antibody.

In certain embodiments of the aAPCs disclosed herein, the liposome has a diameter between 30 nm and 2 μm. In certain embodiments of the aAPCs disclosed herein, the liposome has a diameter between 50 nm and 600 nm. In certain embodiments of the aAPCs disclosed herein, the liposome has a diameter between 100 nm and 400 nm.

In certain embodiments, the present disclosure provides a population of aAPCs disclosed herein. In certain embodiments of the population of aAPCs disclosed herein, the liposomes of the population have a mean diameter between 30 nm and 2 μm and a size distribution of 5% to 50%. In certain embodiments of the population of aAPCs disclosed herein, the mean diameter is between 50 nm and 600 nm. In certain embodiments of the population of aAPCs disclosed herein, the mean diameter is between 100 nm and 400 nm.

In certain embodiments, the present disclosure provides a composition comprising a population of T cells and a population of artificial antigen presenting cells (aAPCs), wherein each aAPC comprises a liposome comprising a phospholipid and a stimulatory ligand displayed on the outer surface of the liposome. In certain embodiments of the compositions disclosed herein, the liposome comprises a mixture of phospholipid and functionalized lipid.

In certain embodiments of the compositions disclosed herein, a ratio of phospholipid to functionalized lipid in the mixture is between 10,000:1 and 25:1. In certain embodiments of the compositions disclosed herein, the ratio is between 1000:1 and 50:1. In certain embodiments of the compositions disclosed herein, the ratio is between 100:1 and 50:1.

In certain embodiments of the compositions disclosed herein, the phospholipid is selected from the group consisting of phosphatidic acid (phosphatidate) (PA), phosphatidylethanolamine (cephalin) (PE), phosphatidylcholine (lecithin) (PC), phosphatidylserine (PS), a phosphoinositide, phosphatidylinositol (PI), phosphatidylinositol phosphate (PIP), phosphatidylinositol bisphosphate (PIP2), phosphatidylinositol triphosphate (PIP3), ceramide phosphorylcholine (Sphingomyelin) (SPH), ceramide phosphorylethanolamine (Sphingomyelin) (Cer-PE), and a combination thereof. In certain embodiments of the compositions disclosed herein, the liposome comprises 18:1 palmitoyl-2-oleoyl-sn-glycero-3-phosphocholine (POPC) and/or 1-palmitoyl-2-oleoyl-sn-glycero-3-phosphoethanolamine (POPE). In certain embodiments of the compositions disclosed herein, the functionalized lipid comprises a biotin moiety, a N-hydroxysuccinimide (NHS) moiety, a sulfo-NHS moiety, a nitrilotriacetic acid (NTA)-nickel, a maleimide moiety, or a N-benzylguanine. In certain embodiments of the compositions disclosed herein, the functionalized lipid is a 1-oleoyl-2-(12-biotinyl-(aminododecanoyl))-sn-glycero-3-phosphoethanolamine (18:1-12:0 Biotin-PE), a 1,2-dipalmitoyl-sn-glycero-3-phosphoethanolamine-N-(biotinyl) (16:0 Biotin-PE), a 1,2-dioleoyl-sn-glycero-3-phosphoethanolamine-N-(biotinyl) (18:1 Biotin-PE), a 1,2-dioleoyl-sn-glycero-3-phosphoethanolamine-N-(cap biotinyl), (18:1 Biotin-Cap-PE), a 1,2-dipalmitoyl-sn-glycero-3-phosphoethanolamine-N-(cap biotinyl) (16:0 Biotin-Cap-PE), a biotin-Phosphatidylethanolamine (biotin-PE), or a biotin-1-palmitoyl-2-oleoyl-sn-glycero-3-phosphoethanolamine (biotin-POPE). In certain embodiments of the compositions disclosed herein, the functionalized lipid is an 18:1 biotin-Cap-PE, a 16:0 biotin-Cap-PE, or a biotin-POPE. In certain embodiments of the compositions disclosed herein, the functionalized lipid is a biotin-POPE.

In certain embodiments of the compositions disclosed herein, the stimulatory ligand is attached to the liposome via the functionalized lipid. In certain embodiments of the compositions disclosed herein, the stimulatory ligand is selected from the group consisting of a CD3 agonist, a CD28 agonist, a Major Histocompatibility Complex (MHC), a peptide-MHC complex, a multimerized neoepitope-HLA complex, CD58, CD86, CD83, 4-1BBL, OX40L, ICOSL (B7H2, B7RP1), CD40L, and an LFA-1. In certain embodiments of the compositions disclosed herein, the stimulatory ligand is a CD3 agonist, a CD28 agonist, or a combination thereof. In certain embodiments of the compositions disclosed herein, the CD3 agonist is an anti-CD3 antibody. In certain embodiments of the compositions disclosed herein, the CD28 agonist is an anti-CD28 antibody. In certain embodiments of the compositions disclosed herein, the anti-CD3 antibody and/or the anti-CD28 antibody is a low-endotoxin azide-free (LEAF) antibody.

In certain embodiments of the compositions disclosed herein, the liposome has a diameter between 30 nm and 2 μm. In certain embodiments of the compositions disclosed herein, the liposome has a diameter between 50 nm and 600 nm. In certain embodiments of the compositions disclosed herein, the liposome has a diameter between 100 nm and 400 nm.

In certain embodiments of the compositions disclosed herein, the composition further comprises a cell growth medium. In certain embodiments of the compositions disclosed herein, the composition further comprises interleukin 7 (IL-7) and interleukin 15 (IL-15). In certain embodiments of the compositions disclosed herein, the population of T cells comprises at least one NeoTCR cell.

In certain embodiments, the present disclosure provides a composition comprising a population of T cells and a population of artificial antigen presenting cells (aAPCs) disclosed herein.

In certain embodiments, the present disclosure provides a method of activating a T cell comprising exposing a T cell to one or more artificial antigen presenting cells (aAPCs), wherein each aAPC comprises a liposome comprising a phospholipid and a stimulatory ligand displayed on the outer surface of the liposome.

In certain embodiments of the methods disclosed herein, the phospholipid is selected from the group consisting of phosphatidic acid (phosphatidate) (PA), phosphatidylethanolamine (cephalin) (PE), phosphatidylcholine (lecithin) (PC), phosphatidylserine (PS), a phosphoinositide, phosphatidylinositol (PI), phosphatidylinositol phosphate (PIP), phosphatidylinositol bisphosphate (PIP2), phosphatidylinositol triphosphate (PIP3), ceramide phosphorylcholine (Sphingomyelin) (SPH), ceramide phosphorylethanolamine (Sphingomyelin) (Cer-PE), and a combination thereof. In certain embodiments of the methods disclosed herein, the liposome comprises 18:1 palmitoyl-2-oleoyl-sn-glycero-3-phosphocholine (POPC) and/or 1-palmitoyl-2-oleoyl-sn-glycero-3-phosphoethanolamine (POPE).

In certain embodiments of the methods disclosed herein, the stimulatory ligand is selected from the group consisting of a CD3 agonist, a CD28 agonist, a Major Histocompatibility Complex (MHC), a peptide-MHC complex, a multimerized neoepitope-HLA complex, CD58, CD86, CD83, 4-1BBL, OX40L, ICOSL (B7H2, B7RP1), and CD40L. In certain embodiments of the methods disclosed herein, the stimulatory ligand is a CD3 agonist, a CD28 agonist or a combination thereof. In certain embodiments of the methods disclosed herein, the CD3 agonist is an anti-CD3 antibody. In certain embodiments of the methods disclosed herein, the CD28 agonist is an anti-CD28 antibody. In certain embodiments of the methods disclosed herein, the anti-CD3 antibody and/or the anti-CD28 antibody is a low-endotoxin azide-free (LEAF) antibody.

In certain embodiments of the methods disclosed herein, the method further comprises mixing a population of T cells with a population of aAPCs. In certain embodiments of the methods disclosed herein, the liposomes of the population of aAPCs have a mean diameter between 30 nm and 2 μm and a size distribution of 5 to 50%. In certain embodiments of the methods disclosed herein, the mean diameter is between 30 nm and 400 nm. In certain embodiments of the methods disclosed herein, the mean diameter is approximately 200 nm. In certain embodiments of the methods disclosed herein, the mixture comprises aAPCs and T cells in a ratio of between 5:1 (aAPCs:T cells) and 5000:1. In certain embodiments of the methods disclosed herein, the T cell is a NeoTCR cell.

In certain embodiments, the present disclosure provides a method of manufacturing a T cell therapy product comprising exposing a population of T cells to a population of artificial antigen presenting cells (aAPCs), wherein each aAPC comprises a liposome comprising a phospholipid and a stimulatory ligand displayed on the outer surface of the liposome.

In certain embodiments of the methods disclosed herein, the method further comprises gene editing at least one T cell of the population of T cells. In certain embodiments of the methods disclosed herein, the gene editing comprises electroporating the population of T cells with a dual ribonucleoprotein species of CRISPR-Cas9 nucleases bound to guide RNA sequences, wherein each species targets an endogenous TCRα locus and/or an endogenous TCRβ locus. In certain embodiments of the methods disclosed herein, the exposing occurs prior to the gene editing. In certain embodiments of the methods disclosed herein, the gene editing is non-viral. In certain embodiments of the methods disclosed herein, the population of T cells comprises one or more NeoTCR cells.

In certain embodiments, the present disclosure provides a method of treating a patient in need thereof with a T cell therapy. In certain embodiments of the methods disclosed herein, the T cell therapy is obtained the methods of manufacturing disclosed herein.

### EXAMPLES

The following are examples of methods and compositions of the invention. It is understood that various other embodiments may be practiced, given the general description provided above.

### Example 1. Evaluation of Available Activation Agents

Six activation agents were compared for suitability for use in the manufacture of NeoTCR Products. The activation agents included four commercially available products: (a) TRANSACT™ (colloidal polymeric nanomatrix conjugated to humanized CD3 and CD28 agonists, Miltenyi Biotec), (b) CLOUDZ™ (12-100 μm diameter microspheres composed of an alginate-based hydrogel, derivatized with fully humanized anti-CD3 and anti-CD28 antibodies, R&D Systems), (c) IMMUNOCULT™ (anti-human CD3 monospecific antibody complex and anti-human CD28 monospecific antibody complex, Stemcell Technologies), and (d) ImmunoCult+CD2. In addition, T cells were exposed to comPACT tetramers (a streptavidin core bound to four biotinylated comPACT proteins) and comPACT-dextran conjugates (streptavidin coated dextran bound to biotinylated comPACT proteins), described in greater detail in U.S. Pat. No. 10,875,905, incorporated herein by reference in its entirety). TransAct and Cloudz also were tested in the presence and absence of IL2 to determine if IL2 would improve T cell activation and lead to improved cell proliferation and physiology.

Edited T cells were prepared as previously described in U.S. Pat. No. 10,584,357. Briefly, CD8 and CD4 positive T cells were enriched from peripheral blood mononuclear cells (PBMCs) isolated from blood by apheresis, by positive selection using magnetic beads (Miltenyi) following the manufacturer's protocol. Enriched T cells were stimulated with the test reagent and cultured with media (TexMACS, 3% human serum containing 12.5 ng/mL IL-7 and IL-15 each) for 13 days. TransAct, Cloudz and ImmunoCult were used as directed by the manufacturer. On Day 2, cells were electroporated with a Neo-TCR homologous recombination template for CRISPR/Cas9 mediated insertion of a gene encoding a NeoTCR in the TRAC locus. T cells were cultured in media until Day 13, at which time gene editing efficiency was determined (Table 3).

TABLE 3
Editing Efficiency of Available Activation Agents

| | % of Live Cells | | |
|---|---|---|---|
| | | TCR | Wild |
| aAPC | NeoTCR+ | Knockout | Type |
| TransAct | 14.7 | 23.7 | 61.6 |
| Cloudz | 13.9 | 54.1 | 30.9 |
| Dextran | 7.99 | 7.12 | 84.7 |
| Tetramer | 4.14 | 4.61 | 91.2 |
| ImmunoCult | 15.7 | 28.4 | 56.0 |
| ImmunoCult + IL2 | 7.69 | 26.1 | 66.3 |

While Cloudz and ImmunoCult activation did promote gene editing, the gene editing efficiency was specific to and skewed toward CD8 T cells (Table 4).

TABLE 4
Activation of CD4+ and CD8+ T cells

| | Total Edited Cells | | % of Live Cells | |
|---|---|---|---|---|
| aAPC | CD4 | CD8 | CD4 | CD8 |
| TransAct | 1.90 × 10⁷ | 2.86 × 10⁷ | 52.4 | 47.6 |
| TransAct + IL-2 | n.d. | n.d. | 44.4 | 55.6 |
| Cloudz | 3.05 × 10⁶ | 6.93 × 10⁷ | 6.54 | 93.5 |
| Cloudz + IL-2 | n.d. | n.d. | 5.59 | 94.4 |
| Dextran | 1.09 × 10⁷ | 1.32 × 10⁶ | 85.0 | 15.0 |
| Tetramer | 5.74 × 10⁶ | 1.26 × 10⁶ | 80.7 | 19.3 |
| ImmunoCult | 4.05 × 10⁶ | 3.20 × 10⁷ | 29.5 | 70.5 |
| ImmunoCult + CD2 | 2.94 × 10⁵ | 1.69 × 10⁶ | 35.8 | 64.2 |

Accordingly, Cloudz and ImmunoCult are not good options for cell therapies that desire effective gene editing of both the CD4 and CD8 T cells. Furthermore, Cloudz is a polymer of approximately 12-100 μm in diameter and removal of such a polymer prior to electroporation (in order to promote efficient gene editing) and/or from the final product (a final cell therapy product that is designed to be infused into a patient preferably has such polymers removed) is a non-trivial task that is time and resource intensive.

### Example 2. T Cell Tolerance of POPC Liposomes

Liposomes were designed to mimic antigen presenting cells (APCs). The liposomes serve as a fluid membrane platform with curvature and stiffness similar to that of living membranes and a surface display of anti-CD3 and anti-CD28 antibodies on the liposomes provide the signals for receptors including the T-cell receptor CD3 complex, and co-stimulatory receptors CD28 on naïve cells.

The experiments described in this example further examine the tolerance of enriched primary CD4/CD8 cells to varying concentrations of 18:1 palmitoyl-2-oleoyl-sn-glycero-3-phosphocholine (POPC) liposomes at ratios of 10:1, 100:1 or 1000:1 liposomes:cells for thirteen days. Stock lipids were solubilized in chloroform. In a borosilicate vial, lipids were mixed by volume in excess chloroform to achieve a desired lipid composition. Using an inert gas, the bulk chloroform solvent is evaporated off to yield a thin lipid film. Any residual chloroform in the lipid film is driven off further in a desiccator under vacuum overnight. The dried lipids were hydrated in room temperature culture media without additives for at least 20 minutes. Liposome formation was achieved by (1) extrusion through track-etched membranes of known pore sizes or (2) sonication.

Peripheral blood mononuclear cells (PBMCs), isolated from blood, were cultured with media. The following day, CD8 and CD4 positive T cells were enriched by positive selection using magnetic beads (Miltenyi) following the manufacturer's protocol and sixteen wells of a 24-well G-Rex® (gas-permeable rapid expansion) plate were seeded with 7.15×106 CD4 and CD8 cells and provided with fresh media (TexMACS Media, 3% hABs, IL-7, IL-15) and TransAct on Days 0 and 8. Liposomes were provided on Days 2 and 8. Viability and count of the T cells were assessed via acridine orange and DAPI staining with a commercial cell counter on Day 0, Day 2, Day 8 and Day 13.

TABLE 5
Tolerance to Liposome Concentration

| liposomes: | % Viability | | | Total Viable Cells | | |
|---|---|---|---|---|---|---|
| cell | Day 2 | Day 8 | Day 13 | Day 2 | Day 8 | Day 13 |
| 10:1 | 94.7 | 96.3 | 96.7 | 4.92 × 10⁶ | 5.14 × 10⁷ | 7.47 × 10⁷ |
| 100:1 | 94.4 | 95.7 | 96.1 | 4.90 × 10⁶ | 4.25 × 10⁷ | 7.10 × 10⁷ |
| 1000:1 | 94.7 | 96.1 | 96.4 | 4.60 × 10⁶ | 4.76 × 10⁷ | 7.61 × 10⁷ |
| control | 95.1 | 96.3 | 96.5 | 5.13 × 10⁶ | 4.11 × 10⁷ | 7.63 × 10⁷ |

As shown in Table 5, both viability and proliferative capacity of enriched, TransAct activated CD4s/CD8s were not affected by presence of blank aAPCs up to a dosage of 1000 aAPCs/cell.

### Example 3. Titration of CD3 and CD28 Agonists

To examine the impact of surface density of signaling molecules anti-CD3 and anti-CD28, 800 nm diameter liposomes were prepared as described above using POPC with 0%, 0.1%, 1%, 2%, and 4% Biotin CAP-PE, as well as include a mock control (TransAct according to the manufacturer's instructions). αCD3 and αCD28 antibodies were bound to the surface of the liposomes as illustrated in FIG. 1. Briefly, biotin conjugated antibodies specific for CD3 or CD28 (both obtained from Miltenyi) were mixed with streptavidin in a ratio of 3:3:2 αCD3:αCD28: streptavidin (effectively 3:1 antibody molecules per molecule of streptavidin) to form antibody-streptavidin trimers. These trimers were then added in excess to biotin-CAP-PE containing liposomes to generate aAPCs displaying the stimulatory ligands, αCD3 and αCD28. All conditions were run in duplicate.

The viability on Day 2 of aAPC conditions trended slightly downward from 97% to 94% with increasing ligand display, but all were equal or above the viability of the TA control cells and consistent with viability expected from the TA control. All aAPC conditions had cell growth from Day 0 to Day 2; TA control had slight decrease in cell number. Increasing the stimulatory ligand presentation increased expression of CD69 and decreased expression of Ki67 (Table 6). TA control had similar levels of CD69 and Ki67 as 0.1% PE condition. All conditions and TA control had substantially the same expression level of CD25.

TABLE 6
Activation and Proliferation Responsive to αCD3/αCD28 Concentration

| | % cells | % cells | % cells |
|---|---|---|---|
| | expressing | expressing | expressing |
| | CD25 | CD69 | Ki67 |
| 0.1% Biotin CAP PE | 93.8 | 32.4 | 21.7 |
| 1.0% Biotin CAP PE | 89.7 | 46.8 | 9.96 |
| 2.0% Biotin CAP PE | 92.7 | 65.8 | 5.06 |
| 4.0% Biotin CAP PE | 89.4 | 67.1 | 2.02 |
| positive control (TA) | 90.6 | 22.5 | 14.3 |

On Day 8, all conditions, including TA control, had poor viability and poor cell growth. The viability trended downward with increasing stimulatory ligand presentation. Unlike with Cloudz and Immunocult, when activated with the liposomes of the invention, there was little difference in editing efficiency between CD4+ and CD8+ cells (data not shown). The lymphocyte population in FSC vs SSC on flow was very small, and there was no TCR signal on these cells, so this was likely an artifact. On Day 8, the viability and cell count correlated positively with Ki67 expression and negatively with CD69 expression.

The experiment was repeated and an evaluation of a broader aAPC dosing strategy was performed on cells expanding through Day 13. Activation with all aAPCs was roughly similar to that obtained with the positive control, TransAct. (Table 7)

TABLE 7
Viability and Growth at Varying Ligand Density

| % PE | % Viability | | | Total Viable Cells | | | Fold |
|---|---|---|---|---|---|---|---|
| biotin | Day 2 | Day 8 | Day 13 | Day 2 | Day 8 | Day 13 | Expansion |
| 4% | 94.4 | 95.5 | 94.8 | 6.55 × 10⁶ | 1.53 × 10⁷ | 7.57 × 10⁷ | 15.13 |
| 2% | 94.1 | 96.7 | 95.1 | 6.92 × 10⁶ | 3.21 × 10⁷ | 9.59 × 10⁷ | 19.17 |
| 1% | 94.4 | 97.1 | 94.8 | 7.17 × 10⁶ | 3.15 × 10⁷ | 8.56 × 10⁷ | 17.12 |
| 0.1% | 96.1 | 96.4 | 96.2 | 6.61 × 10⁶ | 2.26 × 10⁷ | 7.14 × 10⁷ | 14.28 |
| control | 97.1 | 95.8 | 95.6 | 4.61 × 10⁶ | 1.79 × 10⁷ | 7.36 × 10⁷ | 14.72 |

It was determined that fold expansion was greatest in the 2% POPE aAPC condition. Additional interrogation of the 2% POPE aAPC condition was performed to determine the effect on gene editing. 2% PE aAPC condition shows 50% editing at Day 13 and the greatest number of edited cells.

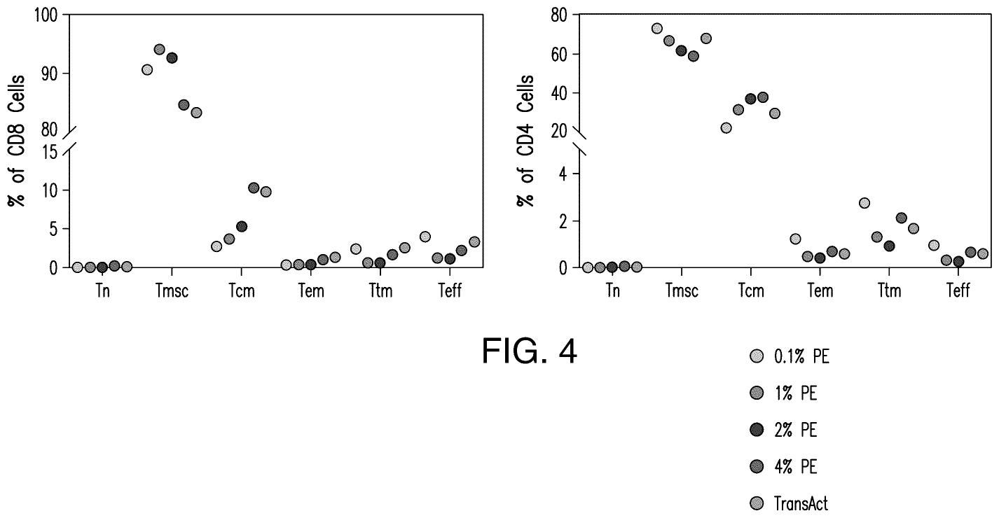

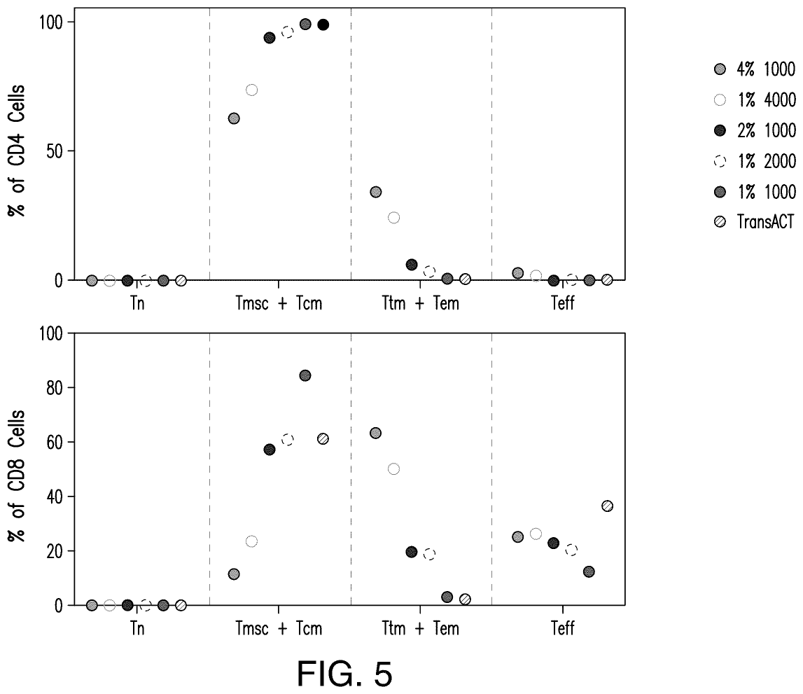

The impact of ligand density on cell phenotype was also examined. As shown in FIG. 4, 0.1-2% POPE show increased % Tmsc and 4% POPE was comparable % Tmsc to TransAct. Also, 1-4% POPE showed lower effector cells, i.e. “older” T cells, compared to TransAct activated cells. Furthermore, increasing ligand density correlated with increased CD4 fraction of the T cell population and therefore distribution of CD4/CD8 T cells may be tunable by adjusting the anti-CD3:anti-CD28 ratio.

Conclusions. Cell expansion significantly improved with increased ligand density on aAPC surface and dosing of aAPCs per cell. 1-4% PE conditions improved NeoTCR+% by >160% over TransAct activated cell population. Additional testing on different electroporation systems can be performed to further optimize the gene editing rates of the aAPC activated cells. Furthermore, the anti-CD3:anti-CD28 molar ratio can be adjusted to optimize the CD4:CD8 T cell populations.

### Example 4. aAPC Diameter for T Cell Activation

CD8 and CD4 positive T cells were enriched from peripheral blood mononuclear cells (PBMCs) isolated from blood by apheresis, by positive selection using magnetic beads (Miltenyi) following the manufacturer's protocol and sixteen wells of a 24-well G-Rex plate were seeded with 7.15×106 CD4 and CD8 cells and provided with fresh media (TexMACS Media, 3% hABs, IL-7, IL-15) and aAPC on Day 0. aAPCs were dosed at 100 liposome/cell (diameter of 30, 100, 200, 400 nm) to activated enriched CD4 and CD8 T cells in duplicate. These aAPCs were present at 1:1 αCD3/αCD28 and a biotin-PE concentration of 1%. On Day 2, the CD4/CD8 T cells were assessed for activation markers prior to electroporation with PACT35-TCR89, a Neo-TCR homologous recombination template for CRISPR/Cas9 mediated insertion of a gene encoding a NeoTCR in the TRAC locus. The media was replenished on Day 8. Gene editing and phenotype state outcome of the expanded cells were assessed on Day 8 and Day 13. Viability and count of the T cells were assessed via acridine orange and DAPI staining with a commercial cell counter on Day 0, Day 2, Day 8 and Day 13.

The viability on Day 2 of aAPC conditions trended slightly downward from 97% to 94% with increasing aAPC size, but all were equal or above the viability of the TA control cells (Table 8). All aAPC conditions had cell growth from Day 0 to Day 2; TA control had slight decrease in cell number.

TABLE 8
Cell Viability as Function of Liposome Size

| liposome | % Viability | | Total Viable Cells | | |
|---|---|---|---|---|---|
| diameter | Day 2 | Day 8 | Day 0 | Day 2 | Day 8 |
| 30 nm | 97.5 | 73.9 | 7.15 × 10⁶ | 1.01 × 10⁷ | 3.58 × 10⁵ |
| 100 nm | 96.8 | 82.2 | 7.15 × 10⁶ | 8.98 × 10⁶ | 6.81 × 10⁵ |
| 200 nm | 95.6 | 86.4 | 7.15 × 10⁶ | 1.32 × 10⁷ | 9.41 × 10⁵ |
| 400 nm | 93.9 | 92.5 | 7.15 × 10⁶ | 8.72 × 10⁶ | 1.83 × 10⁶ |
| Control (TA) | 94.5 | 84.6 | 7.15 × 10⁶ | 6.65 × 10⁶ | 1.08 × 10⁶ |

The 30 nm aAPC condition had similar levels of expression of CD25, CD69, and Ki67 as the unstained control (same donor), likely pointing to insufficient stimulatory ligand to activate the cells (Table 9). aAPC conditions with aAPCs larger than 30 nm showed comparable levels of CD25 as TA-activated control. Increasing the aAPC size increased the expression of CD69 on T cells. In aAPC conditions with aAPCs larger than 30, had comparable or higher expression of CD69 as TA control. All aAPC conditions save 30 nm had higher Ki67 expression than TA control. The expression of CD25, CD69 and Ki67 did not substantially differ between CD4+ and CD8+ cells (data not shown). Similarly, gene editing efficiency did not show any trend relative to liposome size (data not shown).

TABLE 9
Activation and Proliferation Responsive to aAPC size

| | % of cells expressing | | |
|---|---|---|---|
| Liposome diameter | CD25 | CD69 | Ki67 |
| 30 nm | 39.6 | 9.09 | 3.37 |
| 100 nm | 86.1 | 21.1 | 15.3 |
| 200 nm | 94.2 | 27.8 | 23.0 |
| 400 nm | 94.3 | 38.0 | 22.2 |
| positive control (TA) | 90.6 | 22.5 | 14.3 |
| negative control | 34.1 | 8.58 | 3.48 |

Based on the considerations above, it was determined that 200 nm diameter was optimal to enable separation and purification of the T cells from the aAPCs and that 2% or 4% ligand surface coverage was optimal for activation.

### Example 5. Agonist Loading of aAPCs for T Cell Activation

To assess separating the αCD3/αCD28 antibodies onto different liposomes (200 nm), separate αCD3 and αCD28 trimers were generated and bound to aAPCs for a total of 100 liposomes/cell (50 liposomes/cell of each species). Both coupled and uncoupled ligand presentation conditions had better cell growth than TA control from Day 0-2; there were 20% more viable cells in coupled versus uncoupled conditions (data not shown). There were no significant differences in any activation marker between the two conditions. There were slightly higher cell expansion and total number of edited cells in the coupled condition on Day 8, but no significant impact on % NeoTCR+ or % KO between the two conditions.

The results presented above suggest a dependence of T cell activation on aAPC size and ligand presentation modality and density. Those results also point to overstimulation hindering effective activation states of enriched CD4 and CD8 T cells, as measured on process Day 2. In the present study, the effect of lower stimulatory ligand dosage via two avenues was investigated: (1) lower surface density and (2) lower aAPC dosage per T cell. To this end, a large scale (6-well) iteration of 0.01-1% PE in aAPC sizes of 200 nm was performed. aAPC doses ranging from 10 aAPCs/cell and 100 aAPCs/cell were titrated.

CD8 and CD4 positive T cells were enriched from peripheral blood mononuclear cells (PBMCs) isolated from blood by apheresis, by positive selection using magnetic beads (Miltenyi) following the manufacturer's protocol and sixteen wells of a 24-well G-Rex plate were seeded with 7.15×10⁷ CD4 and CD8 cells and provided with fresh media (TexMACS Media, 3% hABs, IL-7, IL-15) and aAPC on Day 0. On Day 2, the CD4/CD8 T cells were assessed for activation markers prior to electroporation with PACT35-TCR89, a Neo-TCR homologous recombination template for CRISPR/Cas9 mediated insertion of a gene encoding a NeoTCR in the TRAC locus. The media was replenished on Day 8. Gene editing and phenotype state outcome of the expanded cells were assessed on Day 8 and Day 13. Viability and count of the T cells were assessed via acridine orange and DAPI staining with a commercial cell counter Day 0, Day 2, Day 8 and Day 13. Activation was assessed on Day 2. T cell phenotype and exhaustion were assessed on Day 8 and Day 13.

At all but the lowest levels of stimulatory ligand, the aAPCs of the invention induced expression of the activation markers, CD25 and CD69, to levels similar to that of the positive control. As seen, previous exposure to aAPCs induced expression of the proliferation marker, Ki67, but at levels lower than the positive control. As with the activation markers, all but the lowest levels of stimulatory ligand showed similar levels of expression of the Ki67.

TABLE 10
Activation and Proliferation

| | | % of cells expressing | | |
|---|---|---|---|---|
| % Biotin PE | APC:cell | CD25 | CD69 | Ki67 |
| 0.01 | 10 | 71.9 | 12.9 | 7.0 |
| 0.01 | 100 | 91.6 | 17.1 | 22.1 |
| 0.1 | 10 | 90.8 | 14.7 | 21.7 |
| 0.1 | 100 | 94.3 | 17.7 | 23.4 |
| 1.0 | 10 | 94.2 | 16.7 | 23.2 |
| 1.0 | 100 | 94.8 | 16.5 | 24.7 |

| | | | |
|---|---|---|---|
| positive control (TA) | 96.4 | 16.1 | 33.3 |

The Ki67 expression was higher in CD4 T cells and lower in CD8 T cells for TA conditions compared to those activated with aAPCs (Table 11). The aAPC condition with the lowest stimulatory ligands (0.01% PE at 10 aAPCs/cell) had lower expression of CD25, CD69, and Ki67 than other conditions.

TABLE 11
Activation and Proliferation of CD4+ and CD8+ T Cells

| | | % CD4+ | | | % CD8+ | | |
|---|---|---|---|---|---|---|---|
| % | APC: | cells expressing | | | cell expressing | | |
| biotin PE | cell | CD25 | CD69 | Ki67 | CD25 | CD69 | Ki67 |
| 0.01 | 10 | 76.7 | 11.0 | 4.65 | 56.1 | 23.3 | 16.5 |
| 0.01 | 100 | 94.1 | 15.9 | 19.3 | 86.9 | 26.7 | 35.2 |
| 0.1 | 10 | 93.4 | 14.0 | 18.7 | 84.5 | 24.0 | 35.1 |
| 0.1 | 100 | 94.7 | 18.3 | 20.0 | 95.5 | 23.5 | 38.9 |
| 1.0 | 10 | 94.6 | 17.3 | 19.7 | 95.2 | 22.8 | 38.9 |
| 1.0 | 100 | 95.9 | 17.6 | 22.3 | 94.9 | 21.9 | 36.9 |

| | | | | | | |
|---|---|---|---|---|---|---|
| Positive control | 98.5 | 18.2 | 35.0 | 94.7 | 19.5 | 32.1 |
| (TA) | | | | | | |

Gene editing was examined in relation to titration of anti-CD3 and anti-CD28 surface display and aAPC size on T cell activation and engagement. Conditions with higher moles of stimulatory ligand had higher NeoTCR expression compared to those with lower stimulatory ligand, but similar levels of Knock Out (data not shown). By Day 8, aAPCs had no effect on CD4:CD8 ratio, which was approximately 3:1 for all conditions tested, including the TA control.

Activation markers were analyzed to determine if there was an effect of aAPC size on T cell activation and engagement if the amount of stimulatory ligand was held constant. CD69 and CD25 were used as the activation markers and Ki67 was used as the proliferation marker. aAPCs (0.1% PE) were selected between the range of 200-800 nm with varying doses of 6-100 aAPCs/cell. Moles of stimulatory ligands were kept constant in all conditions (3.02 picomoles each of αCD3 antibody and αCD28 antibody per assay or approximately 25,000 stimulatory ligands per T cell). Similarly, the size of the vesicle and dose (APCs) were inversely varied such that total liposomal surface area was constant (1.26×10 7 nm 2). Expression levels of CD25, CD69 and Ki67 were independent of size and aAPC/cell dosing at equimolar agonist levels (Table 12). With moles of stimulatory ligand held constant, aAPC size/dosage did not affect neoTCR expression (Table R).

TABLE 12
Activation and Proliferation with Equimolar Ligand

| | | % CD4+ | | | % CD8+ | | |
|---|---|---|---|---|---|---|---|
| | | cells expressing | | | cells expressing | | |
| Diameter | (APC:cell) | CD25 | CD69 | Ki67 | CD25 | CD69 | Ki67 |
| 200 nm | 100 | 94.7 | 18.3 | 20.0 | 95.5 | 23.5 | 38.9 |
| 400 nm | 25 | 95.1 | 17.4 | 20.7 | 95.9 | 22.7 | 40.1 |
| 800 nm | 6.25 | 95.3 | 17.5 | 21.1 | 96 | 22.4 | 40.5 |

| | | | | | | |
|---|---|---|---|---|---|---|
| positive control | 98.5 | 18.2 | 35.0 | 94.7 | 19.5 | 32.1 |
| (TA) | | | | | | |

These experiments show that it is the total amount of stimulatory ligand, not size or aAPC dosing, that affects activation and editing of enriched CD4/CD8 T cells.

### Example 6. Liposome Resistance to Fusion with T Cells

The aAPCs of the invention are liposome constructs that consist of lipids, such as POPC and biotin-POPE lipids with conjugated streptavidin trimers of different activation signaling molecules. These aAPCs can be used for the activation of CD4/CD8 T cells. Liposomes, however, have the potential to fuse with the patient cells during ligand interaction. To establish whether liposomes would fuse with patient cells or to what extent that fusion occurred, Texas Red DHPE, a lipid conjugated to Texas Red, was used as a marker for lipid fusion with lymphocytes. anti-CD3/anti-CD28 trimers were generated and bound to biotinylated lipids on the liposomes (1% Texas Red, 1% biotin-POPE in POPC).

Fusion was assessed using flow cytometry. Day −1 (minus one) thawed enriched CD4/CD8 T cells were plated in complete media and rested for 24 hours. Day 0, Texas Red liposomes (TR-liposomes with αCD3/αCD28 (1% PE)) were produced at a diameter of 800 nm and added to culture at a dose of 10 liposomes per cell. The timepoints to be assessed were Day 0, Day 2 pre-centrifugation, Day 2 post-centrifugation, and Day 2 post-electroporation after rest. On Day 0, five wells were plated with 10M cells and aAPCs added at 10 aAPCs/cell. After two hours on Day 0, one well was collected and diluted in 1% BSA/PBS, and run on flow to check for red signal and presence of aAPCs. To have a baseline reading on liposomes, mean fluorescent intensity (MFI) of Tx-Red liposomes was also measured.

At Day 2, samples were assessed to test for effects of electroporation on aAPC fusion. The cells were resuspended into the media and an aliquot pre-centrifugation sample was taken along with supernatant from culture before resuspension, and then post resuspension sample. Two of the remaining cultures were resuspended and centrifuged at 100 g for 10 min. For the D2 post-centrifugation, the supernatant was collected and the pellet was resuspended in 1% BSA/PBS. For the post-electroporation sample, the supernatant was removed, and the pellet resuspended in 100 μL P3 Primary Cell Nucleofector Solution (Lonza) buffer and electroporated in an X cuvette. After 10 minutes, the cells were returned to media and cultured for 2 hours at 37° C., after which the cells were collected for flow analysis.

To determine whether the aAPCs interact with the enriched T cells from D0 to D2, the T cells and aAPCs were monitored over a 2-day period. On D0 four wells of 25000 T cells were plated with 10 aAPCs/cell. Two wells received complete aAPCs, while the other two wells received blank aAPCs (with Texas Red, without biotin PE) as a negative control for stimulatory ligands. Images were taken every 2 hours for two days to assess T cell:aAPC interaction.

The timeline for the processing of the cells is as follows: 1) Day −1: Dry lipids for aAPCs [1% TR, 1% Biotin-PE], thaw and rest cells at 10M/well; 2) Day 0: Add to rested cells 10 aAPCs/cell, diameter of 800 nm with αCD3/αCD28; assess D0 fusion; and 3) Day 2: Electroporation in X cuvette conditions 4,5 and assess fusion 2 hrs post-electroporation; assess D0 fusion.

TransAct activated cells cluster together, which is most visible at 48 hrs (data not shown). This phenomenon is not seen to the same extent in the aAPC activated conditions. This could be due to (1) lack of stimulatory ligands necessary for LFA-1 ICAM-1 upregulation necessary for self-clustering or (2) steric blocking of LFA-1 ICAM-1 interaction by aAPCs.

On Day 0, only the cells four hours post-addition of aAPCs had any TxRed signal. This suggests that the T cells are engaging with the aAPCs by four hours in culture before settling.

On Day 2, only the culture supernatant sample has TxRed signal. This demonstrates that aAPCs of an initial size of 800 nm do not settle in culture. The cells pre-centrifugation have no TxRed signal, suggesting lack of fusion or engagement with aAPCs post-settling by Day 2. Post-centrifugation and post-electroporation cells also had no TxRed signal. This suggests that centrifugation does not promote fusion of aAPCs with the T cells and is sufficient to clear aAPCs pre-electroporation.

### Example 7. aAPC Dose and Ligand Density Driven T Cell Clustering

Summary Zumwalde et al., J. Immunol. 2013 191:3681-3693, have shown the LFA-1/ICAM-1 interaction mediates homotypic adhesion between activated T cells, because T cells express both LFA-1 and ICAM-1. Such homotypic aggregates are a hallmark of efficient T cell activation in vitro and T cell clusters have also been observed following antigen-specific T cell activation in vivo. ICAM-1 is an early T cell activation marker that is regulated by IL-12 and that the disruption of T cell clusters enhances development of CD8 T cell effector functions by regulating both access of antigen to activated CD8 T cells, as well as the expression levels of CTLA-4 and eomesodermin.

T cell clustering can be monitored with the Sartorius IncuCyte instrument that takes periodic images of cell cultures. aAPCs were dosed at varying aAPC:T-cell ratios and images were acquired every 2 hours to monitor clustering events. The experiment included TransAct stimulated and unstimulated T cells, as positive and negative controls, respectively, for comparative analysis. aAPC stimulation did initiate T cell clustering events, however, that extent of clustering was far less compared to TransAct bead activated cell cultures.

Rather than carrying out extensive 13-process day studies as described above, T cell clustering was used as a proxy for assessing and potentially narrowing the optimal range of aAPC dose and ligand density construct required to test at large scale. Additionally, these measurements illuminated whether the clustering process is an essential precursor for sufficient T cell activation prior to electroporation at Day2.

The experiments described in this example describe a screen of an αCD3/αCD28 ligand density of 0.1-4% of aAPC surface and also the dose range of 100-5000 aAPCs/cell.

CD8 and CD4 positive T cells were enriched from peripheral blood mononuclear cells (PBMCs) isolated from blood by apheresis, by positive selection using magnetic beads (Miltenyi) following the manufacturer's protocol. Conditions were plated in triplicate in a 96-well plate format. Each well was seeded with 100,000 T cells and aAPCs (in total volume of 10 μL) and monitored over 48 hours with images captured every 2 hours. TransAct was used in accordance with manufacturer's instructions. All liposomes were prepared as approximately 200 nm in diameter. Readouts were taken on Day 0 (enrichment of CD4/CD8 T cells, Cell Counts and Viability, plating for IncuCyte study) and Day 2 (image analysis). All cells were cultured in TexMACs 3% HS+IL7 and IL15 (both at 12.5 ng/mL).

To confirm the non-toxicity of aAPCs in this experimental model, cells were grown in in culture in the presence of POPC liposomes (0% PE) at concentrations of 0, 10, 100, and 1000 liposomes/cell and activated with TransAct. There was no difference in the growth rates across all concentrations of liposomes/cell, including the absence of liposomes. This confirms that the aAPCs are not toxic.

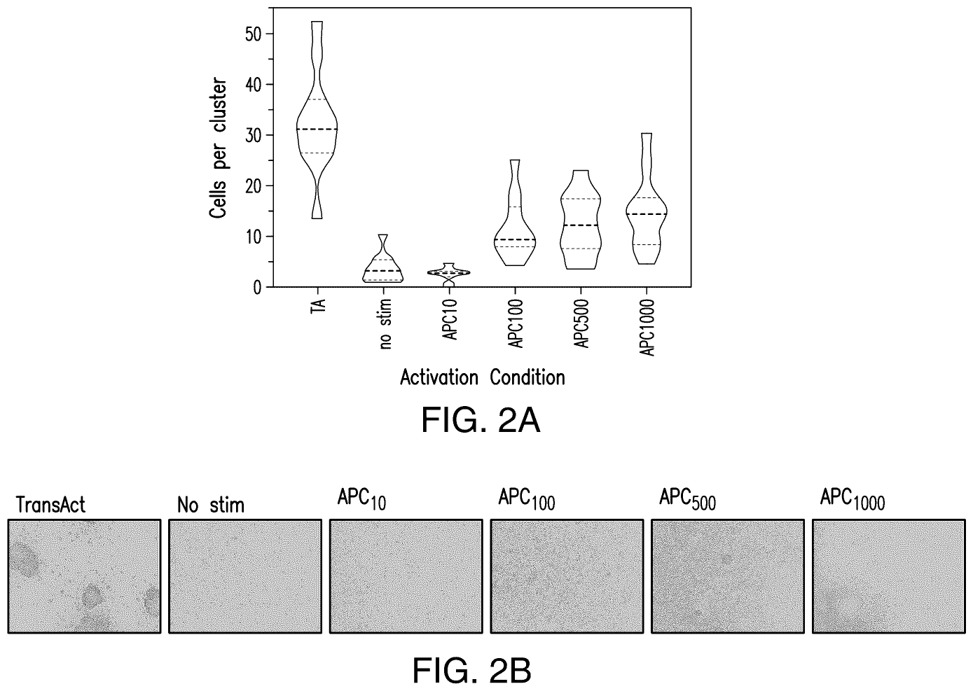

T cell clustering was monitored during the activation phase (FIGS. 2A and 2B). For these experiments, 25,000 T cells were plated in 96 well plates with aAPCs including between 0.1% and 4% PE and at a dosage of between 100:1 and 5000:1 aAPC:cell (each condition plated in triplicate). Images were acquired every 6 hours during the activation period. ICAM-1 dependent homotypic clustering of T cells during activation was monitored.

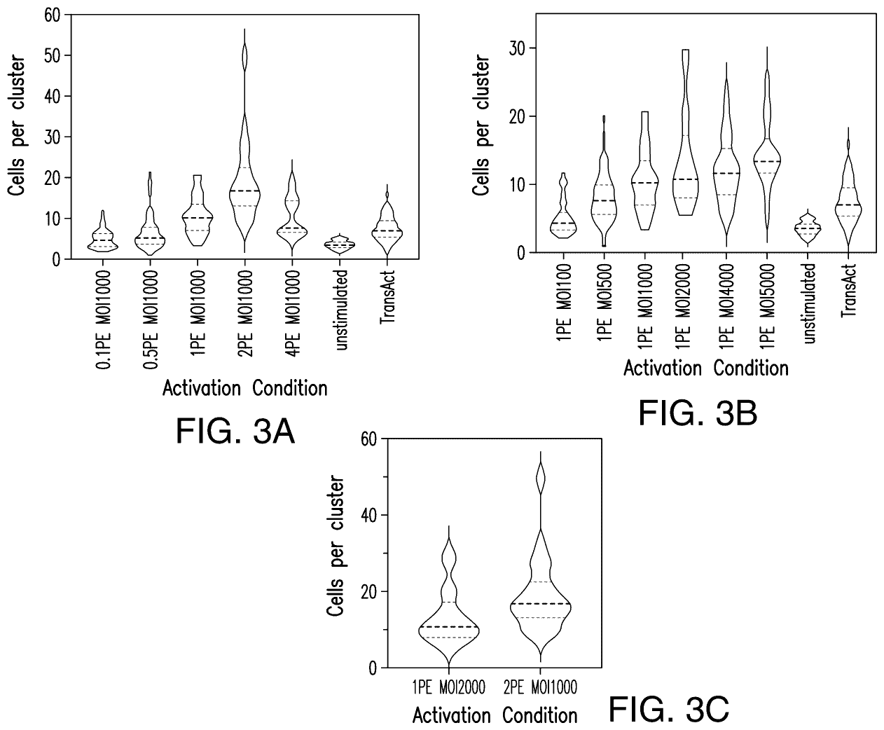

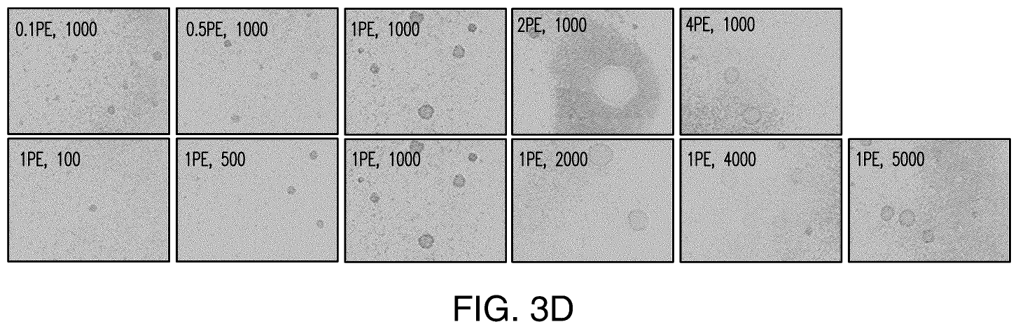

Experiments were performed to determine if T cell clustering would be improved with increased aAPC dosage. All conditions were evaluated on IncuCyte for activation induced clustering. FIG. 3A illustrates clustering as a function of CD3 and CD28 agonist, with the dosage held constant at 1000:1 aAPC:cell. The amount of clustering increased with increasing ligand density from 0.1% PE to 2% PE but dropped substantially at 4% PE. FIG. 3B illustrates the impact of dosage on clustering, with all liposomes containing 1% PE in POPC. Clustering increased with dosage from 100 aAPC:cell to 1000 aAPC:cell at which point providing additional liposomes had little effect. FIG. 3C demonstrates that aAPCs having 2% PE at a dose of 1000:1 induce slightly more clustering than aAPCs having 1% PE at a dose of 2000:1. Images of activated cells are provided in FIG. 3D.

### Example 8. Large-Scale aAPCs—Ligand Density with Optimized Large-Scale Cuvette-Based Electroporation

Summary. The use of aAPCs to activate enriched CD4/CD8 T cells for electroporation using an optimized large-scale cuvette-based electroporation system (1 mL cuvettes) was evaluated. Above, it was demonstrated the use of aAPCs as activators for CD4s and CD8s at small scale with comparable knock-in and improved knock-out compared to the TransAct control. It was also demonstrated that electroporation efficiencies were improved using a large scale 1 mL cuvette optimized electroporation system. The experiments described in this example tested three different aAPC ligand surface densities compared to TransAct activation in the large scale 1 mL cuvette optimized electroporation system.

CD8 and CD4 positive T cells were enriched from peripheral blood mononuclear cells (PBMCs) isolated from blood by apheresis, by positive selection using magnetic beads (Miltenyi) following the manufacturer's protocol. 71.5M cells were activated per each condition in a 6-well G-Rex plate. Ligand density ranged from 1-4% PE and dosing ranged from 1000 to 4000 aAPC:cell while maintaining a constant mean diameter of approximately 200 nm. On Day 2, 50M cells from each condition were electroporated with PACT035 TCR089 in P3 buffer. The study was run in TexMACS, supplemented with IL-7 and IL-15, for thirteen days. Media was replenished on Day 8. Gene editing and phenotype state outcome of the expanded cells were assessed on Day 8 and Day 13. Viability and count of the T cells were assessed via acridine orange and DAPI staining with a commercial cell counter Day 2, Day 8 and Day 13.

TABLE 13
Results of Large-Scale Activation with aAPCs

| Condition | | Cell Counts | | | Fold | Gene Editing | |
|---|---|---|---|---|---|---|---|
| % PE | aAPCs/cell | Day 2 | Day 8 | Day 13 | Expansion | % NeoTCR | Edited Cells |
| 4% | 1000 | 5.0 × 10⁷ | 1.19 × 10⁸ | 5.74 × 10⁸ | 11.5 | 73.2 | 4.20 × 10⁸ |
| 2% | 1000 | 5.0 × 10⁷ | 1.35 × 10⁸ | 6.26 × 10⁸ | 12.5 | 68.9 | 4.32 × 10⁸ |
| 1% | 1000 | 5.0 × 10⁷ | 1.59 × 10⁸ | 6.35 × 10⁸ | 12.7 | 59.0 | 3.75 × 10⁸ |
| 1% | 4000 | 5.0 × 10⁷ | 1.27 × 10⁸ | 5.84 × 10⁸ | 11.7 | 68.5 | 4.00 × 10⁸ |
| 1% | 2000 | 5.0 × 10⁷ | 1.47 × 10⁸ | 6.39 × 10⁸ | 12.8 | 67.4 | 4.31 × 10⁸ |

| | | | | | | |
|---|---|---|---|---|---|---|
| TransAct | 5.0 × 10⁷ | 2.02 × 10⁸ | 5.26 × 10⁸ | 10.5 | 53.2 | 2.80 × 10⁸ |

The aAPC evaluation at Day 13 showed that the highest stimulatory ligand dosage results in highest editing but slightly lower expansion than lower stimulatory ligands (Table 13). TransAct had lowest expansion and editing. aAPC activated conditions had at most 3.6% WT on Day 13, while TransAct condition had 14.3% (data not shown). It was also shown that all conditions had between 13-20% CD4+ cells and that increased dosage of stimulatory ligands increases Ttm/Tem populations and reduces Tmsc+Tcm populations (FIG. 6).

It is possible to successfully activate enriched CD4/CD8 T cells with aAPCs at intermediate scale with high NeoTCR+ expression and low % of wild-type cells on Day 13. As stimulatory ligand dosage increases, increased Ttm/Tem populations were observed. With lowest ligand dosage, improved Tmsc/Tcm population in CD8 T cells compared to TransAct was observed.

### Example 9. Dynamic Ranges of Anti-CD3 and Anti:CD28 Antibody Ratios on the aAPC Surface

Summary. The experiments performed in this example were designed to determine the effects of anti-CD3:anti-CD28 ratios on the surface of the aAPCs.

Results. To confirm that it is possible to titrate ligand display on aAPCs, liposomes were prepared as described above, but biotinylated fluorophores (AlexaFluor 488-biotinylated, AlexaFluor 594-biotinylated) were used in place of the biotinylated stimulatory ligands. Geometric mean of individual MFIs of varying constructs showed that it was possible to resolve and create different aAPC species (Table 14).

TABLE 14
Liposomes Displaying Fluorophores

| Biotin- | Biotin- | Ratio | MFI | MFI | Norm | Norm | Ratio |
|---|---|---|---|---|---|---|---|
| AF488 | AF594 | (expected) | AF488 | AF594 | AF488 | AF594 | (observed) |
| 1 | 1 | 1 | 8021 | 2036 | 1 | 1 | 1.00 |
| 1 | 2 | 0.5 | 6028 | 3175 | 0.8 | 1.6 | 0.48 |
| 1 | 5 | 0.2 | 3142 | 4524 | 0.4 | 2.2 | 0.18 |
| 1 | 10 | 0.1 | 1477 | 4162 | 0.2 | 2.0 | 0.09 |
| 2 | 1 | 2 | 12619 | 1460 | 1.6 | 0.7 | 2.19 |
| 5 | 1 | 5 | 18213 | 757 | 2.3 | 0.4 | 6.11 |
| 10 | 1 | 10 | 18964 | 178 | 2.4 | 0.1 | 27.04 |

CD8 and CD4 positive T cells were enriched from peripheral blood mononuclear cells (PBMCs) isolated from blood by apheresis, by positive selection using magnetic beads (Miltenyi) following the manufacturer's protocol.

The next question was whether it would be possible to identify an optimal ligand ratio for activation and priming for electroporation. CD8 and CD4 positive T cells were enriched from peripheral blood mononuclear cells (PBMCs) isolated from blood by apheresis, by positive selection using magnetic beads (Miltenyi) following the manufacturer's protocol and sixteen wells of a 24-well G-Rex plate were seeded with 7.15×10⁶ CD4 and CD8 cells and provided with fresh media (TexMACS Media, 3% hABs, IL-7, IL-15) and aAPC on Day 0. Cell media was exchanged on Day 8. It was determined that the range of αCD28 display did not appear to affect cell expansion and viability. In contrast, the range of αCD3 display did have an effect. Specifically, 5:1 showed increased cell expansion and there was improved cell health with increasing αCD3: αCD28 ratio up 10:1 (Table 15).

TABLE 15
Stimulatory Ligand Ratios

| Ligand Ratio | | Live Cells | | | Gene Editing |
|---|---|---|---|---|---|
| αCD3 | αCD28 | Day 2 | Day 8 | Day 13 | % NeoTCR |
| 1 | 1 | 6.19 × 10⁷ | 1.72 × 10⁸ | 7.96 × 10⁸ | 35.7 |
| 1 | 2 | 6.28 × 10⁷ | 1.40 × 10⁸ | 7.32 × 10⁸ | 37.4 |
| 1 | 5 | 6.81 × 10⁷ | 1.51 × 10⁸ | 7.42 × 10⁸ | 42.7 |
| 2 | 1 | 4.86 × 10⁷ | 2.00 × 10⁸ | 7.76 × 10⁸ | 33.4 |
| 5 | 1 | 6.38 × 10⁷ | 2.00 × 10⁸ | 1.14 × 10⁸ | 32.1 |
| 10 | 1 | 6.06 × 10⁷ | 1.41 × 10⁸ | 7.54 × 10⁸ | 30.4 |
| 1 LEAF | 1 LEAF | 5.63 × 10⁷ | 1.56 × 10⁸ | 1.11 × 10⁸ | 48.8 |

| | | | | |
|---|---|---|---|---|
| TransAct | 6.26 × 10⁷ | 1.77 × 10⁸ | 6.42 × 10⁸ | 36.5 |

To assess the impact of ligand ratios on gene editing, cells were cultured with aAPC (1% PE, 200 nm diameter, at 1000 aAPC:cell, with varying ligand ratios), for 44-48 hours prior to electroporation on Day 2. On Day 2, 50 million cells cultured under each condition were nucleofected and then cultured in fresh media supplemented with aAPC. Media was exchanged on Day 8. Increasing anti-CD28 surface display improved editing efficiency such that a 5× increase in αCD28 resulted in 23% increase in NeoTCR+ cells. In contrast, increasing anti-CD3 surface display decreased editing efficiency such that there was a 17% decrease in NeoTCR+ with 10-fold increase in αCD3 stimulation (Table 15; activation markers were measured but data is not shown).

Experiments were also performed to determine if low endotoxin, azide-free (LEAF) formulations of ligands (Miltenyi RUO antibodies (clones OKT3, 15E8) and Biolegend LEAF antibodies (clones OKT3, 28.2)) affected cell expansion and/or gene editing efficiencies. The data showed that the aAPCs with LEAF activators improve cell expansion but also increase the rate of media consumption in aAPC activated T cells due to accumulated 2× greater lactate due 2× greater cell expansion. It was further shown that even at 1% surface ligand density (aAPC activated T cells resulted in 25% greater NeoTCR+%) and that aAPC activated T cells yielded 2-fold greater total edited cells. In summary, it was shown that Biolegend LEAF antibodies at a 1% surface area coverage of co-stimulatory ligands, aAPCs drive higher electroporation efficiency and result in greater number of edited cells.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. An artificial antigen presenting cell (aAPC) comprising a liposome comprising a phospholipid and a stimulatory ligand displayed on the outer surface of the liposome.

2. The aAPC of claim 1, wherein the stimulatory ligand is selected from the group consisting of a CD3 agonist, a CD28 agonist, a Major Histocompatibility Complex (MHC), a peptide-MHC complex, a multimerized neoepitope-HLA complex, CD58, CD86, CD83, 4-1BBL, OX40L, ICOSL (B7H2, B7RP1), CD40L, and an LFA-1.

3. The aAPC of claim 1 or 2, wherein the liposome comprises a mixture of phospholipid and functionalized lipid.

4. The aAPC of claim 3, wherein a ratio of phospholipid to functionalized lipid in the mixture is between 10,000:1 and 25:1.

5. The aAPC of claim 4, wherein the ratio is between 1000:1 and 50:1.

6. The aAPC of claim 4 or 5, wherein the ratio is between 100:1 and 50:1.

7. The aAPC of any one of claims 1-6, wherein the phospholipid is selected from the group consisting of phosphatidic acid (phosphatidate) (PA), phosphatidylethanolamine (cephalin) (PE), phosphatidylcholine (lecithin) (PC), phosphatidylserine (PS), a phosphoinositide, phosphatidylinositol (PI), phosphatidylinositol phosphate (PIP), phosphatidylinositol bisphosphate (PIP2), phosphatidylinositol triphosphate (PIP3), ceramide phosphorylcholine (Sphingomyelin) (SPH), ceramide phosphorylethanolamine (Sphingomyelin) (Cer-PE), and a combination thereof.

8. The aAPC of any one of claims 1-6, wherein the liposome comprises 18:1 palmitoyl-2-oleoyl-sn-glycero-3-phosphocholine (POPC) and/or 1-palmitoyl-2-oleoyl-sn-glycero-3-phosphoethanolamine (POPE).

9. The aAPC of any one of claims 3-8, wherein the functionalized lipid comprises a biotin moiety, a N-hydroxysuccinimide (NHS) moiety, a sulfo-NHS moiety, a nitrilotriacetic acid (NTA)-nickel, a maleimide moiety, or a N-benzylguanine.

10. The aAPC of any one of claims 3-9, wherein the functionalized lipid is a 1-oleoyl-2-(12-biotinyl-(aminododecanoyl))-sn-glycero-3-phosphoethanolamine (18:1-12:0 Biotin-PE), a 1,2-dipalmitoyl-sn-glycero-3-phosphoethanolamine-N-(biotinyl) (16:0 Biotin-PE), a 1,2-dioleoyl-sn-glycero-3-phosphoethanolamine-N-(biotinyl) (18:1 Biotin-PE), a 1,2-dioleoyl-sn-glycero-3-phosphoethanolamine-N-(cap biotinyl), (18:1 Biotin-Cap-PE), a 1,2-dipalmitoyl-sn-glycero-3-phosphoethanolamine-N-(cap biotinyl) (16:0 Biotin-Cap-PE), a biotin-Phosphatidylethanolamine (biotin-PE), or a biotin-1-palmitoyl-2-oleo yl-sn-glycero-3-phosphoethanolamine (biotin-POPE).

11. The aAPC of claim 10, wherein the functionalized lipid is an 18:1 biotin-Cap-PE, a 16:0 biotin-Cap-PE, or a biotin-POPE.

12. The aAPC of claim 10 or 11, wherein the functionalized lipid is a biotin-POPE.

13. The aAPC of any one of claims 1-12, wherein the stimulatory ligand is attached to the liposome via the functionalized lipid.

14. The aAPC of any one of claims 1-13, wherein the stimulatory ligand is a CD3 agonist, a CD28 agonist, or a combination thereof.

15. The aAPC of claim 14, wherein the CD3 agonist is an anti-CD3 antibody.

16. The aAPC of claim 14, wherein the CD28 agonist is an anti-CD28 antibody.

17. The aAPC of claim 15 or 16, wherein the anti-CD3 antibody and/or the anti-CD28 antibody is a low-endotoxin azide-free (LEAF) antibody.

18. The aAPC of any one of claims 1-17, wherein the liposome has a diameter between 30 nm and 2 μm.

19. The aAPC of claim 18, wherein the liposome has a diameter between 50 nm and 600 nm.

20. The aAPC of claim 18 or 19, wherein the liposome has a diameter between 100 nm and 400 nm.

21. A population of aAPC of any one of claims 1-20.

22. The population of claim 21, wherein the liposomes of the population have a mean diameter between 30 nm and 2 μm and a size distribution of 5% to 50%.

23. The population of claim 22, wherein the mean diameter is between 50 nm and 600 nm.

24. The population of claim 22 or 23, wherein the mean diameter is between 100 nm and 400 nm.

25. A composition comprising a population of T cells and a population of artificial antigen presenting cells (aAPCs), wherein each aAPC comprises a liposome comprising a phospholipid and a stimulatory ligand displayed on the outer surface of the liposome.

26. The composition of claim 25, wherein the liposome comprises a mixture of phospholipid and functionalized lipid.

27. The composition of claim 26, wherein a ratio of phospholipid to functionalized lipid in the mixture is between 10,000:1 and 25:1.

28. The composition of claim 27, wherein the ratio is between 1000:1 and 50:1.

29. The composition of claim 27 or 28, wherein the ratio is between 100:1 and 50:1.

30. The composition of any one of claims 25-29, wherein the phospholipid is selected from the group consisting of phosphatidic acid (phosphatidate) (PA), phosphatidylethanolamine (cephalin) (PE), phosphatidylcholine (lecithin) (PC), phosphatidylserine (PS), a phosphoinositide, phosphatidylinositol (PI), phosphatidylinositol phosphate (PIP), phosphatidylinositol bisphosphate (PIP2), phosphatidylinositol triphosphate (PIP3), ceramide phosphorylcholine (Sphingomyelin) (SPH), ceramide phosphorylethanolamine (Sphingomyelin) (Cer-PE), and a combination thereof.

31. The composition of any one of claims 25-30, wherein the liposome comprises 18:1 palmitoyl-2-oleoyl-sn-glycero-3-phosphocholine (POPC) and/or 1-palmitoyl-2-oleoyl-sn-glycero-3-phosphoethanolamine (POPE).

32. The composition of any one of claims 26-31, wherein the functionalized lipid comprises a biotin moiety, a N-hydroxysuccinimide (NHS) moiety, a sulfo-NHS moiety, a nitrilotriacetic acid (NTA)-nickel, a maleimide moiety, or a N-benzylguanine.

33. The composition of any one of claims 26-32, wherein the functionalized lipid is a 1-oleoyl-2-(12-biotinyl-(aminododecanoyl))-sn-glycero-3-phosphoethanolamine (18:1-12:0 Biotin-PE), a 1,2-dipalmitoyl-sn-glycero-3-phosphoethanolamine-N-(biotinyl) (16:0 Biotin-PE), a 1,2-dioleoyl-sn-glycero-3-phosphoethanolamine-N-(biotinyl) (18:1 Biotin-PE), a 1,2-dioleoyl-sn-glycero-3-phosphoethanolamine-N-(cap biotinyl), (18:1 Biotin-Cap-PE), a 1,2-dipalmitoyl-sn-glycero-3-phosphoethanolamine-N-(cap biotinyl) (16:0 Biotin-Cap-PE), a biotin-Phosphatidylethanolamine (biotin-PE), or a biotin-1-palmitoyl-2-oleoyl-sn-glycero-3-phosphoethanolamine (biotin-POPE).

34. The composition of claim 33, wherein the functionalized lipid is an 18:1 biotin-Cap-PE, a 16:0 biotin-Cap-PE, or a biotin-POPE.

35. The composition of claim 33 or 34, wherein the functionalized lipid is a biotin-POPE.

36. The composition of any one of claims 25-35, wherein the stimulatory ligand is attached to the liposome via the functionalized lipid.

37. The composition of any one of claims 25-36, wherein the stimulatory ligand is selected from the group consisting of a CD3 agonist, a CD28 agonist, a Major Histocompatibility Complex (MHC), a peptide-MHC complex, a multimerized neoepitope-HLA complex, CD58, CD86, CD83, 4-1BBL, OX40L, ICOSL (B7H2, B7RP1), CD40L, and an LFA-1.

38. The composition of any one of claims 25-37, wherein the stimulatory ligand is a CD3 agonist, a CD28 agonist, or a combination thereof.

39. The composition of claim 38, wherein the CD3 agonist is an anti-CD3 antibody.

40. The composition of claim 38, wherein the CD28 agonist is an anti-CD28 antibody.

41. The composition of claim 39 or 40, wherein the anti-CD3 antibody and/or the anti-CD28 antibody is a low-endotoxin azide-free (LEAF) antibody.

42. The composition of any one of claims 25-41, wherein the liposome has a diameter between 30 nm and 2 μm.

43. The composition of claim 42, wherein the liposome has a diameter between 50 nm and 600 nm.

44. The composition of claim 42 or 43, wherein the liposome has a diameter between 100 nm and 400 nm.

45. The composition of any one of claims 25-44 further comprising a cell growth medium.

46. The composition of any one of claims 25-45 further comprising interleukin 7 (IL-7) and interleukin 15 (IL-15).

47. The composition of any one of claims 25-46, wherein the population of T cells comprises at least one NeoTCR cell.

48. A composition comprising a population of T cells and a population of artificial antigen presenting cells (aAPCs) of any one of claims 21-25.

49. A method of activating a T cell comprising exposing a T cell to one or more artificial antigen presenting cells (aAPCs), wherein each aAPC comprises a liposome comprising a phospholipid and a stimulatory ligand displayed on the outer surface of the liposome.

50. The method of claim 50, wherein the phospholipid is selected from the group consisting of phosphatidic acid (phosphatidate) (PA), phosphatidylethanolamine (cephalin) (PE), phosphatidylcholine (lecithin) (PC), phosphatidylserine (PS), a phosphoinositide, phosphatidylinositol (PI), phosphatidylinositol phosphate (PIP), phosphatidylinositol bisphosphate (PIP2), phosphatidylinositol triphosphate (PIP3), ceramide phosphorylcholine (Sphingomyelin) (SPH), ceramide phosphorylethanolamine (Sphingomyelin) (Cer-PE), and a combination thereof.

51. The method of claim 49 or 50, wherein the liposome comprises 18:1 palmitoyl-2-oleoyl-sn-glycero-3-phosphocholine (POPC) and/or 1-palmitoyl-2-oleoyl-sn-glycero-3-phosphoethanolamine (POPE).

52. The method of any one of claims 49-51, wherein the stimulatory ligand is selected from the group consisting of a CD3 agonist, a CD28 agonist, a Major Histocompatibility Complex (MHC), a peptide-MHC complex, a multimerized neoepitope-HLA complex, CD58, CD86, CD83, 4-1BBL, OX40L, ICOSL (B7H2, B7RP1), and CD40L.

53. The method of any one of claims 49-52, wherein the stimulatory ligand is a CD3 agonist, a CD28 agonist or a combination thereof.

54. The method of claim 53, wherein the CD3 agonist is an anti-CD3 antibody.

55. The method of claim 53, wherein the CD28 agonist is an anti-CD28 antibody.

56. The method of claim 54 or 55, wherein the anti-CD3 antibody and/or the anti-CD28 antibody is a low-endotoxin azide-free (LEAF) antibody.

57. The method of any one of claims 49-56, further comprising mixing a population of T cells with a population of aAPCs.

58. The method of claim 57, wherein the liposomes of the population of aAPCs have a mean diameter between 30 nm and 2 μm and a size distribution of 5% to 50%.

59. The method of claim 58, wherein the mean diameter is between 30 nm and 400 nm

60. The method of claim 59, wherein the mean diameter is approximately 200 nm.

61. The method of any one of claim 57-60, wherein the mixture comprises aAPCs and T cells in a ratio of between 5:1 (aAPCs:T cells) and 5000:1.

62. The method of any one of claims 49-61, wherein the T cell is a NeoTCR cell.

63. A method of manufacturing a T cell therapy product comprising exposing a population of T cells to a population of artificial antigen presenting cells (aAPCs), wherein each aAPC comprises a liposome comprising a phospholipid and a stimulatory ligand displayed on the outer surface of the liposome.

64. The method of claim 63, further comprising gene editing of at least one T cell of the population of T cells.

65. The method of claim 64, wherein the gene editing comprises electroporating the population of T cells with a dual ribonucleoprotein species of CRISPR-Cas9 nucleases bound to guide RNA sequences, wherein each species targets an endogenous TCRα locus and/or an endogenous TCRβ locus.

66. The method of claim 63 or 64, wherein the exposing occurs prior to the gene editing.

67. The method of any one of claims 63-66, wherein the gene editing is non-viral.

68. The method of any one of claims 63-67, wherein the population of T cells comprises one or more NeoTCR cells.

69. A method of treating a patient in need thereof with a T cell therapy, wherein the T cell therapy is obtained by the method of any one of claims 63-69.
